(19) United States
(12) Patent Application Publication
(10) Pub. No.: US 2009/0069287 A1
(43) Pub. Date: Mar. 12, 2009

(54) SUBSTITUTED AZACYCLOALKANES USEFUL FOR TREATING CNS CONDITIONS

(75) Inventors: James Gorham Boyd, Mystic, CT (US); Todd William Butler, Salem, CT (US); Brian Thomas O'Neill, Haddam, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC

(21) Appl. No.: 12/094,175

(22) PCT Filed: Nov. 10, 2006
(86) PCT No.: PCT/IB2006/003318

§ 371 Date: May 19, 2008

Related U.S. Application Data
(60) Provisional application No. 60/739,262, filed on Nov. 22, 2005.

(51) Int. Cl. A61K 31/397; C07D 211/58; A61K 31/451; C07D 205/04; A61K 31/506; C07D 403/12

(52) U.S. Cl. 514/210.17; 546/224; 514/329; 514/256; 544/322; 548/953

(57) ABSTRACT

The invention relates to substituted azacycloalkaπe compounds useful in treating conditions of the Central Nervous System (CNS); a pharmaceutical composition comprising same; a method of treating such conditions and of treating conditions in which inhibition of beta-secretase is indicated.

### FIELD OF THE INVENTION

The invention relates to substituted azacycloalkane compounds useful in treating conditions of the Central Nervous System (CNS); a pharmaceutical composition comprising same; a method of treating such conditions and of treating conditions in which inhibition of beta-secretase is indicated.

### BACKGROUND OF THE INVENTION

Conditions affecting the Central Nervous System include neurodegenerative conditions such as Alzheimer's Disease. Various of these conditions are typified by physical changes in the brain. For example, certain such pathologies are evidenced by the presence of neurofibrillary tangles and/or plaque deposits which, as they progress, cause cognitive, motor, sensory and other impairments on multiple fronts. Commonly, said plaques are comprised principally of beta-amyloid-a highly aggregative protein that tends to accumulate, forming insoluble deposits that can ultimately cause cellular injury and death. Beta-amyloid (Aβ) derives from an amyloid precursor protein (APP), which is a transmembrane protein existing in several isoforms, the more prevalent of which contain 695, 714, 751 or 771 amino acids (denoted APP₈₉₅, APP₇₁₄, APP₇₅₁ and APP₇₇₁ respectively). The generation of beta-amyloid is due to the sequential cleavage of APP by various proteases: beta-secretase cleaves APP at an N terminus while gamma-secretase cleaves it at a C terminus. The resulting fragment is beta-amyloid-a protein of 38, 40, 42 or 43 amino acids (denoted Aβ₁₋₃₈, Aβ₁₋₄₀, Aβ₁₋₄₂, Aβ₁₋₄₃ respectively). This fragment is released into the extracellular space where it accumulates with other such insoluble fragments to form the proteinaceous deposits aforesaid that are neuronally toxic.

Amongst treatment strategies under investigation for such conditions is the development of compounds that will among other things effectively inhibit beta-secretase and or its processing of APP to reduce the formation of Aβ and ameliorate plaque deposition and related pathogenesis.

### SUMMARY OF THE INVENTION

The invention is to a compound of Formula I as more particularly defined hereinbelow:

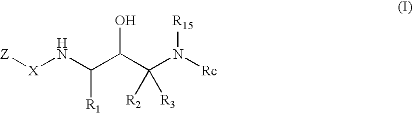

The invention is further to a pharmaceutical composition comprising the compound of Formula (I); and methods of treating a CNS condition and/or a condition in which the inhibition

of beta-secretase is indicated comprising administering to a patient in need of such treatment or in whom such inhibition is indicated, an effective amount of said compound.

Referring to Formula I:

Z is hydrogen, (C₃-C₇cycloalkyl)₀₋₁(C₁-C₈ alkyl), (C₃-C₇ cycloalkyl)₀₋₁(C₂-C₆ alkenyl), (C₃-C₇ cycloalkyl)₀₋₁(C₂-C₆alkynyl), —O—(C₁-C₆) alkyl, —O—(C₂-C₆) alkenyl, —(C₁-C₆) alkyl(C₆-C₁₀)aryl, —(C₂-C₆) alkylene(C₆-C₁₀)aryl or (C₃-C₇ cycloalkyl)-, wherein each of said groups is independently optionally substituted with 1, 2 or 3 R_{Z} groups;

wherein R_{Z} at each occurrence is independently halogen, —OH, —SH, —CN, —CF₃, —OCF₃, (C₁-C₆)alkoxy, (C₃-C₇)cycloalkyl, (C₃-C₇)cycloalkoxy or —NR₁₀₀R₁₀₁;

where R₁₀₀ and R₁₀ₗ are independently H, (C₁-C₆)alkyl, phenyl, CO(C₁-C₆)alkyl or SO₂ (C₁-C₆)alkyl;

wherein X is —(C═O)— or —(SO₂)—;

wherein R₁ is (C₁-C₁₀)alkyl optionally substituted with 1, 2, or 3 groups independently selected from halogen, —OH, ═O, —SH, —CN, —CF₃, —OCF₃, —(C₃-C₇)cycloalkyl, —(C₁-C₄)alkoxy, —NR₁₀₀R₁₀ₗ, (C₆-C₁₀)aryl, (5 to 9 member)heteroaryl and (5 to 9 member) heterocyclo, wherein each aryl group is optionally substituted with 1, 2 or 3 R₅₀ groups;

wherein R₅₀ is selected from halogen, OH, SH, CN, —CO—(C₁-C₄)alkyl, —NR₇R₆, —S(O)₁₋₂—(C₁-C₄ alkyl), (C₁-C₆)alkyl, (C₂-C₆)alkenyl, (C₂-C₆)alkynyl, (C₁-C₆)alkoxy and (C₃-C₆) cycloalkyl; wherein the alkyl, alkenyl, alkynyl, alkoxy and cycloalkyl groups are optionally substituted with 1 or 2 substituents independently selected from the group consisting of (C₁-C₄)alkyl, halogen, OH, —NR₅R₆, CN, (C₁-C₄)haloalkoxy, NR₇R₈ and (C₁-C₄)alkoxy;

wherein R₅ and R₆ are independently H or (C₁-C₆)alkyl; or

wherein R₅ and R₆ and the nitrogen to which they are attached form a 5 or 6 membered heterocycloalkyl ring; and

wherein R₇ and R₈ are independently selected from the group consisting of H; —(C₁-C₄)alkyl optionally substituted with 1, 2, or 3 groups independently selected from the group consisting of —OH, —NH₂ and halogen; —(C₃-C₆)cycloalkyl; —(C₁-C₄ alkyl)-O—(C₁-C₄ alkyl); —(C₂-C₄)alkenyl; and —(C₂-C₄)alkynyl;

wherein each heteroaryl of R₁ is optionally substituted with 1 or 2 R₅₀ groups; wherein each heterocyclo group of R₁ is optionally substituted with 1 or 2 groups that are independently R₅₀ or ═O;

wherein R₂ and R₃ are independently selected from H; —F; —(C₁-C₆)alkyl optionally substituted with a substituent selected from the group consisting of —F, —OH, —CN, —CF₃, (C₁-C₃)alkoxy and —NR₅R₆; or R₂ and R₃ are independently selected from —(CH₂)₀₋₂—R₁₇; —(CH₂)₀₋₂—R₁₈; —(C₂-C₆)alkenyl or —(C₂-C₆)alkynyl, wherein each group is optionally substituted with an independent substituent selected from the group consisting of —F, —OH, —CN, —CF₃, (C₁-C₃)alkoxy; —(CH₂)₀₋₂—(C₃-C₇)cycloalkyl, optionally substituted with an independent substituent selected from the group consisting of —F, —OH, —CN, —CF₃, (C₁-C₃)alkoxy and —NR₅R₆; or

wherein R₂, R₃ and the carbon to which they are attached form a —(C₃-C₇)cycloalkyl ring of three through seven carbon atoms, wherein one carbon atom is optionally replaced by a group selected from —O—, —S—, —SO₂— or —NR₇—;

where R₁₇ at each occurrence is an aryl group selected from phenyl, 1-naphthyl, 2-naphthyl, indanyl, indenyl, dihydronaphthyl and tetralinyl, wherein said aryl groups are optionally substituted with one or two groups that are independently —(C₁-C₃)alkyl; —(C₁-C₄)alkoxy; CF₃; or R₁₇ at each occurrence is —(C₂-C₆)alkenyl or —(C₂-C₆)alkynyl each of which is optionally substituted with one substituent selected from the group consisting of F, OH, (C₁-C₃)alkoxy; or R₁₇ at each occurrence is selected from

-halogen;

—OH;

—CN;

—(C₃-C₇)cycloalkyl;

—CO—(C₁-C₄ alkyl);

—SO₂—(C₁-C₄ alkyl);

where R₁₈ is a heteroaryl group selected from pyridinyl, pyrimidinyl, quinolinyl, indolyl, pyridazinyl, pyrazinyl, isoquinolyl, quinazolinyl, quinoxalinyl, phthalazinyl, imidazolyl, isoxazolyl, oxazolyl, thiazolyl, furanyl, thienyl, pyrrolyl, oxadiazolyl or thiadiazolyl, wherein each of said heteroaryl groups is optionally substituted with one or two groups that are independently —(C₁-C₆)alkyl optionally substituted with one substituent selected from the group consisting of OH, CN, CF₃, (C₁-C₃)alkoxy and —NR₅R₆;

wherein Rc is

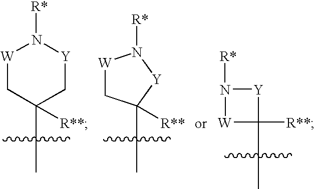

wherein W and Y are each independently —CH₂— or C═O;

wherein R* is

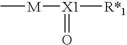

wherein M is —(CH₂)p- or C═O; X1 is C, S═O or is absent; p is 0-3; with the proviso that when M is C═O, X1 is C;

wherein R*₁ is hydrogen, (C₁-C₆)alkyl, (C₁-C₆)alkoxy, hydroxyl (C₁-C₆) alkyl optionally substituted with up to three halogens or OH groups; phenyl; (5 to 9 member)heteroaryl and (5 to 9 member) heterocyclo wherein said heteroaryl is selected from the group consisting of thiazolyl, oxazolyl, 1,2,4-oxadiazolyl, 1,3,4- and 1,2,4-thiadiazolyl, imidazolyl, isoxazolyl, pyridinyl, pyrimidinyl wherein said heteroaryl or heterocyclo is optionally substituted by halogen, (C₁-C₆)alkyl, (C₁-C₆) alkoxy, (C₁-C₆) alkoxyCH₂—, CN, NO₂, CF₃, —NH(C₁-C₆) alkyl, —NH₂, (C₁-C₆)alkyl —CO—NH— or (C₆-C₁₀)aryl(C₁-C₆)alkoxy; said phenyl optionally substituted with (C₁-C₆)alkyl or up to three —(C═O)R₁₅ wherein R*₅ is H, (C₁-C₆)alkyl or OH, NR*₂R*₃ or (C═O)(O)₀₋₁R*₄, wherein R*₂, R*₃ and R*₄ are each independently H or (C₁-C₆)alkyl; and

wherein R** is (C₆-C₁₀)aryl, (C₅-C₉)heteroaryl, (C₁-C₆)alkyl, (C₂-C₆) alkenyl, (C₂-C₆) alkynyl, —O—(C₁-C₆) alkyl, —O—(C₂-C₆) alkenyl or —(C₂-C₆) alkylene-(C₆-C₁₀)aryl;

wherein each (C₆-C₁₀)aryl of R** is phenyl or naphthyl, each (5 to 9 member) heteroaryl ring is optionally fused to a benzo group and contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, chloro, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, (3-8 membered)heterocyclo, hydroxyl(3-8 membered)heterocyclo, and (C₁-C₆)alkoxy-(3-8 membered)heterocyclo, wherein said alkyl, alkoxy and cycloalkyl may be optionally substituted with 1 to 3 halos and wherein each (C₃-C₈)cycloalkyl or heterocyclo moiety may be independently substituted with from one to three (C₁-C₆)alkyl, phenyl or benzyl groups; or

wherein each (C₅-C₉)heteroaryl ring of R** is optionally fused to an imidazo, pyrido, pyrimido, pyrazo, pyridazo, or pyrrolo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing fused heteroaryl rings may optionally be substituted with from one to three substituents independently selected from (C₁-C₈) alkyl, chloro-, bromo-, iodo, fluoro-, halo(C₁-C₈)alkyl, hydroxy(C₁-C₈)alkyl-, (C₁-C₈)alkoxy-(C₁-C₈)alkyl-, —O—(C₁-C₈)alkyl-halo, hydroxy(C₃-C₈)cycloalkyl-, (C₃-C₈)cycloalkoxy-, (C₁-C₈)alkoxy-(C₃-C₈)cycloalkyl-, (5 to 9 member) heterocyclo, hydroxyl (5 to 9 member) heterocyclo and (C₁-C₆)alkoxy-(5 to 9 member)heterocyclo, wherein each (C₃-C₈)cycloalkyl or heterocyclo moiety may be independently substituted with from one to three (C₁-C₆)alkyl or benzyl groups;

with the proviso that when X is —(C═O), Z is methyl, R₁ is difluorobenzyl and R₂ and R₃ are each hydrogen, Rc is

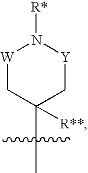

and when
W and Y are each —CH₂— and R** is (C₃-C₄)alkyl substituted phenyl,
R* in combination with M, X1 and R F may not be H, —C₂H₅, (—CH₃), —C₂H₄OH, —C₂H₄CN, —(C═O)NH₂, CH₃—SO₂—, C₂H₅—SO₂—, —H(C═O), —(C═O)CH₃ or —(C═O)CF₃.

In another embodiment, the present invention has the additional proviso that when X is —(C═O), Z is methyl, R₁ is difluorobenzyl and R₂ and R₃ are each hydrogen, Rc is

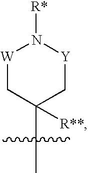

and when
W and Y are each —CH₂ and R** is (C₃-C₄)alkyl substituted phenyl,

R* in combination with M, X1 and R₁ may not be (C₁-C₃) alkyl, hydroxyl (C₁-C₃) alkyl, —CN(C₁-C₃)alkyl, —(C═O)NR₇R₈, (C₁-C₃)alkyl-SO₂—, —(C₁-C₈)alkyl-CHO, —(C═O)(C₁-C₃) alkyl or —(C═O)(C₁-C₃) alkyl wherein said alkyl is substituted with 3-5 fluoro atoms.

The present invention also provides a compound having the structure of Formula Ia:

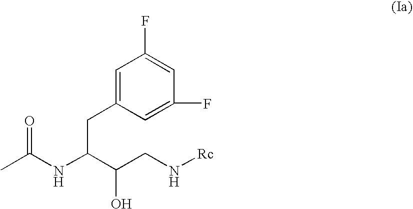

wherein Rc is as hereinabove defined.

A particular practice of the present invention relates to Formula Ia wherein Rc is

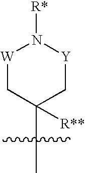

wherein each of W, Y, R* and R** has the same meaning as defined hereinabove.

A preferred embodiment relates to compounds of Formula Ia wherein Rc is as above, wherein W and Y are each —CH₂—; R* is —COOCH₃ or —COO—CH₂-phenyl; and R** is (C₁-C₄) alkyl substituted phenyl.

Another preferred embodiment relates to compounds of Formula Ia wherein W is —CH₂— and Y is C═O; R* is H, (C₁-C₄)alkyl, —CH₂COOH, —CH₂COOCH₃, —CH₂COOCH(CH₃)₂, CH₃CH₂-phenyl or —CH₂—CH₂OH; and R** is (C₁-C₄) alkyl substituted phenyl. Most preferably, the carbon atom at the piperidone ring 4 position is a chiral carbon atom in the S configuration.

Another practice of the present invention relates to Formula Ia wherein Rc is

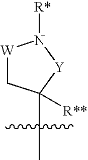

wherein each of W, Y, R* and R** has the same meaning as defined above.

In a preferred embodiment W and Y are each —CH₂—, R** is (C₃-C₄)alkyl substituted phenyl and R* is

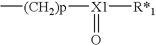

wherein p is 0, X1 is C and R*₁ is CH₃, —OCH₃ or —CH₂—COOCH₃.

In another preferred embodiment X1 is absent and R*₁ is a (5-6 membered)heteroaryl ring. Most preferably R*₁ is 2-thiazolyl or 2-pyrimidinyl.

Another embodiment relates to Formula Ia wherein Rc is

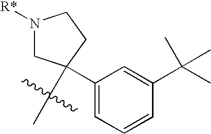

wherein R* is

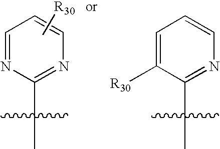

wherein R₃₀ is (C₁-C₆)alkyl, (C₁-C₆) alkoxy, (C₁-C₆)alkylene-O—(C₁-C₆)alkyl, CN, NH₂, NH(C₁-C₆)alkyl, CF₃, NO₂ or halogen.

Another practice of the present invention relates to compounds of Formula Ia wherein Rc is

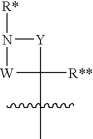

wherein each of W, Y, R* and R** has the same meaning as defined above.

In a preferred embodiment W and Y are each —CH₂—, R** is (C₃-C₄)alkyl substituted phenyl and R* is

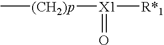

wherein p is 0, X1 is C and R*₁ is —CH₂—CO—OCH₃.

The present invention also provides a process for preparing a compound of the formula

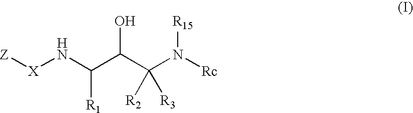

comprising reacting, under conditions effective to form said compound (I), a compound of formula

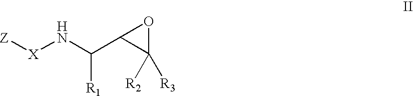

with a compound of formula

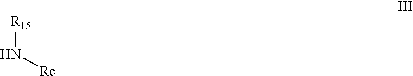

wherein Z, X, R₁, R₂, R₃, Rc and R₁₅ are as defined hereinabove.

Representative compounds of this practice include:
4-[(2R,3S)-3-Acetylamino-4-(3,5-difluoro-phenyl)-2-hydroxy-butylamino]-4-(3-isopropyl-phenyl)-piperidine-1-carboxylic acid methyl ester;
N-{(1S,2R)-1-(3,5-Difluoro-benzyl)-2-hydroxy-3-[1-(2-hydroxy-acetyl)-4-(3-isopropyl-phenyl)-piperidin-4-ylamino]-propyl}-acetamide;
[(2R,3S)-4-[3-Acetylamino-4-(3,5-difluoro-phenyl)-2-hydroxy-butylamino]-4-(3-isopropyl-phenyl)piperidin-1-yl]-oxo-acetic acid;
[(2R,3S)-4-[3-Acetylamino-4-(3,5-difluoro-phenyl)-2-hydroxy-butylamino]-4-(3-isopropyl-phenyl)-piperidin-1-yl]-oxo-acetic add methyl ester;
N-{(1S,2R)-1-(3,5-Difluoro-benzyl)-2-hydroxy-3-[4-(3-isopropyl-phenyl)-1-(2,2,2-trifluoro-ethanesulfonyl)-piperidin-4-ylamino]-propyl}acetamide;
4-[(2R,3S)-3-Acetylamino-4-(3,5-difluoro-phenyl)-2-hydroxy-butylamino]-4-(3-isopropyl-phenyl)-piperidine-1-carboxylic acid methylamide;
4-[(2R,3S)-3-Acetylamino-4-(3,5-difluoro-phenyl)-2-hydroxy-butylamino]-4-(3-isopropyl-phenyl)-piperidine-1-carboxylic acid ethyl ester;
4-[(2R,3S)-3-Acetylamino-4-(3,5-difluoro-phenyl)-2-hydroxy-butylamino]-4-(3-tert-butyl-phenyl)-piperidine-1-carboxylic acid benzyl ester;
4-[(2R,3S)-3-Acetylamino-4-(3,5-difluoro-phenyl)-2-hydroxy-butylamino]-4-(3-isopropyl-phenyl)-piperidine-1-carboxylic acid benzyl ester;
4-[(2R,3S)-3-Acetylamino-4-(3,5-difluoro-phenyl)-2-hydroxy-butylamino]-4-(3-tert-butyl-phenyl)-piperidine-1-carboxylic acid methyl ester;
N-[(1S,2R)-3-[4-(3-tert-Butyl-phenyl)-1-butyryl-piperidin-4-ylamino]-1-(3,5-difluoro-benzyl)-2-hydroxy-propyl]-acetamide;
N-[(1S,2R)-3-[4-(3-tert-Butyl-phenyl)-1-(3-methyl-butyryl)-piperidin-4-ylamino]-1-(3,5-difluoro-benzyl)-2-hydroxy-propyl]-acetamide;
4-[(2R,3S)-3-Acetylamino-4-(3,5-difluoro-phenyl)-2-hydroxy-butylamino]-4-(3-tert-butyl-phenyl)piperidine-1-carboxylic acid dimethylamide;
[(2R,3S)-4-[3-Acetylamino-4-(3,5-difluoro-phenyl)-2-hydroxy-butylamino]-4-(3-isopropyl-phenyl)-2-oxo-piperidin-1-yl]-acetic acid methyl ester;
[(2R,3S,4R)-4-[3-Acetylamino-4-(3,5-difluoro-phenyl)-2-hydroxy-butylamino]-4-(3-isopropyl-phenyl)-2-oxo-piperidin-1-yl]-acetic acid methyl ester;
[(2R,3S)-4-[3-Acetylamino-4-(3,5-difluoro-phenyl)-2-hydroxy-butylamino]-4-(3-isopropyl-phenyl)-2-oxo-piperidin-1-yl]-acetic acid;
[(2R,3S,4R)-4-[3-Acetylamino-4-(3,5-difluoro-phenyl)-2-hydroxy-butylamino]-4-(3-isopropyl-phenyl)-2-oxo-piperidin-1-yl]-acetic acid;
[(2R,3S,4S)-4-[3-Acetylamino-4-(3,5-difluoro-phenyl)-2-hydroxy-butylamino]-4-(3-isopropyl-phenyl)-2-oxo-piperidin-1-yl]-acetic acid methyl ester;
[(2R,3S,4S)-4-[3-Acetylamino-4-(3,5-difluoro-phenyl)-2-hydroxy-butylamino]-4-(3-isopropyl-phenyl)-2-oxo-piperidin-1-yl]-acetic acid;
N-{(1S,2R)-1-(3,5-Difluoro-benzyl)-3-[1-ethyl-4-(3-isopropyl-phenyl)-2-oxo-piperidin-4-ylamino]-2-hydroxy-propyl}-acetamide;
N-{(1S,2R,4R)-1-(3,5-Difluoro-benzyl)-3-[1-ethyl-4-(3-isopropyl-phenyl)-2-oxo-piperidin-4-ylamino]-2-hydroxy-propyl}acetamide;
N-{(1S,2R,4S)-1-(3,5-Difluoro-benzyl)-3-[1-ethyl-4-(3 isopropyl-phenyl)-2-oxo-piperidin-4-ylamino]-2-hydroxy-propyl}-acetamide;
N-[(1S,2R)-3-[1-Benzyl-4-(3 isopropyl-phenyl)-2-oxo-piperidin-4-ylamino]-1-(3,5-difluoro-benzyl)-2-hydroxy-propyl]-acetamide;
N-[(1S,2R,4S)-3-[1-Benzyl-4-(3-isopropyl-phenyl)-2-oxo-piperidin-4-ylamino]-1-(3,5-difluoro-benzyl)-2-hydroxy-propyl]-acetamide;
N-[(1S,2R,4R)-3-[1-Benzyl-4-(3-isopropyl-phenyl)-2-oxo-piperidin-4-ylamino]-1-(3,5-difluorobenzyl)-2-hydroxy-propyl]-acetamide;
N-{(1S,2R)-1-(3,5-Difluoro-benzyl)-2-hydroxy-3-[1-(2-hydroxy-ethyl)-4-(3-isopropyl-phenyl)-2-oxo-piperidin-4-ylamino]-propyl}acetamide;
N-{(1S,2R,4R)-1-(3,5-Difluoro-benzyl)-2-hydroxy-3-[1-(2-hydroxy-ethyl-4-(3-isopropyl-phenyl)-2-oxo-piperidin-4-ylamino]-propyl}acetamide;
N-{(1S,2R,4S)-1-(3,5-Difluoro-benzyl)-2-hydroxy-3-[1-(2-hydroxy-ethyl)-4-(3-isopropyl-phenyl)-2-oxo-piperidin-4-ylamino]-propyl}acetamide;
N-{(1S,2R)-1-(3,5-Difluoro-benzyl)-2-hydroxy-3-[4-(3-isopropyl-phenyl)-2-oxo-piperidin-4-ylamino]-propyl}acetamide;
N-{(1S,2R,4S)-1-(3,5-Difluoro-benzyl)-2-hydroxy-3-[4-(3-isopropyl-phenyl)-2-oxo-piperidin-4-ylamino]-propyl}-acetamide;
N-{(1S,2R,4R)-1-(3,5-Difluoro-benzyl)-2-hydroxy-3-[4-(3-isopropyl-phenyl)-2-oxo-piperidin-4-ylamino]-propyl}acetamide;
N-(1S,2R)-[3-[1-(2-tert-Butoxy-ethyl)-4-(3-isopropyl-phenyl)2-oxo-piperidin-4-ylamino]-1-(3,5-difluoro-benzyl)-2-hydroxy-propyl]-acetamide;
N-(1S,2R,4S)-[3-[1-(2-tert-Butoxy-ethyl)-4-(3-isopropyl-phenyl)-2-oxo-piperidin-4-ylamino]-1-(3,5-difluoro-benzyl)-2-hydroxy-propyl]-acetamide;
N-(1S,2R,4R)-[3-[1-(2-tert-oxy-ethyl)-4-(3-isopropyl-phenyl)-2-oxo-piperidin-4-ylamino]-1-(3,5-difluoro-benzyl)-2-hydroxy-propyl]-acetamide;
(2R,3S)-[4-[3-Acetylamino-4-(3,5-difluoro-phenyl)-2-hydroxy-butylamino]-4-(3-isopropyl-phenyl)-2-oxo-piperidin-1-yl]-acetic acid isopropyl ester;
(2R,3S,4R)-[4-[3-Acetylamino-4-(3,5-difluoro-phenyl)-2-hydroxy-butylamino]-4-(3-isopropyl-phenyl)-2-oxo-piperidin-1-yl]-acetic acid isopropyl ester;
(2R,3S,4S)-[4-[3-Acetylamino-4-(3,5-difluoro-phenyl)-2-hydroxy-butylamino]-4-(3-isopropyl-phenyl)-2-oxo-piperidin-1-yl]-acetic acid isopropyl ester;
N-[(1S,2R)-3-[1-Acetyl-3-(3-isopropyl-phenyl)-pyrrolidin-3-ylamino]-1-(3,5-difluoro-benzyl)-2-hydroxy-propyl]-acetamide;
3-[(2S,3R)-3-Acetylamino-4-(3,5-difluoro-phenyl)-2-hydroxy-butylamino]-3-(3-isopropyl-phenyl)-pyrrolidine-1-carboxylic acid methyl ester;
3-[(2S,3R)-3-Acetylamino-4-(3,5-difluoro-phenyl)-2-hydroxy-butylamino]-3-(3-isopropyl-phenyl)-pyrrolidine-1-carboxylic acid methyl ester;
N-{(1S,2R)-1-(3,5-Difluoro-benzyl)-2-hydroxy-3-[3-(3-isopropyl-phenyl)-1-methanesulfonyl-pyrrolidin-3-ylamino]-propyl}acetamide;
N-{(1S,2R)-1-(3,5-Difluoro-benzyl)-2-hydroxy-3-[3-(3-isopropyl-phenyl)-1-methanesulfonyl-pyrrolidin-3-ylamino]-propyl}acetamide;
3-[(2S,3R)-3-[3-Acetylamino-4-(3,5-difluoro-phenyl)-2-hydroxy-butylamino]-3-(3-isopropyl-phenyl)-pyrrolidin-1-yl]-3-oxo-propionic acid methyl ester;
3-[(2S,3R)-3-[3-Acetylamino-4-(3,5-difluoro-phenyl)-2-hydroxy-butylamino]-3-(3-isopropyl-phenyl)-pyrrolidin-1-yl]-3-oxo-propionic acid methyl ester;
N-{(1S,2R)-1-(3,5-Difluoro-benzyl)-2-hydroxy-3-[3-(3 isopropyl-phenyl)-1-pyridin-2-yl-pyrrolidin-3-ylamino]-propyl}acetamide;
N-[(1S,2R)-3-[1-Benzooxazol-2-yl-3-(3-isopropyl-phenyl)-pyrrolidin-3-ylamino]-1-(3,5-difluoro-benzyl)-2-hydroxy-propyl]-acetamide;
N-{(1S,2R)-1-(3,5-Difluoro-benzyl(2-hydroxy-3-[3-(3-isopropyl-phenyl)-thiazol-2-yl-pyrrolidin-3-ylamino]-propyl}acetamide;
N-{(1S,2R)-1-(3,5-Difluoro-benzyl)-2-hydroxy-3-[3-(3-isopropyl-phenyl)-1-pyrimidin-2-yl-pyrrolidin-3-ylamino]-propyl}acetamide;
N-[(1S,2R)-3-[1-(5-Bromo-pyrimidin-2-yl)-3-(3-tert-butyl-phenyl)-pyrrolidin-3-ylamino]-1-(3,5-difluoro-benzyl)-2-hydroxy-propyl]-acetamide;
N-[(1S,2R)-3-[3-(3-tert-Butyl-phenyl)-1-(4-methoxy-pyrimidin-2-yl)-pyrrolidin-3-ylamino]-1-(3,5-difluoro-benzyl)-2-hydroxy-propyl]-acetamide;
3-[(2R,3S)-3-Acetylamino-4-(3,5-difluoro-phenyl)-2-hydroxy-butylamino]-3-(3-isopropyl-phenyl)-azetidine-1-carboxylic acid benzyl ester;

3-[3-[(2R,3S)-3-Acetylamino-4-(3,5-difluoro-phenyl)-2-hydroxy-butylamino]-3(3-isopropyl-phenyl)-azetidin-1-yl]-3-oxo-propionic acid methyl ester;
3-[(2R,3S)-3-Acetylamino-4-(3,5-difluoro-phenyl)-2-hydroxy-butylamino]-3-(3-isopropyl-phenyl)-azetidine-1-carboxylic acid methyl ester;
N-[(1S,2R)-3-[1-Acetyl-3-(3-isopropyl-phenyl)-azetidin-3-ylamino]-1-(3,5-difluoro-benzyl)-2-hydroxy-propyl]-acetamide;
N-[(1S,2R)-3-[4-(3-tert-Butyl-phenyl)-1-pyrimidin-2-yl-piperidin-4-ylamino]-1-(3,5-difluoro-benzyl)-2-hydroxy-propyl]-acetamide; and
N-[(1S,2R)-3-[4-(3-tert-Butyl-phenyl)-1-thiazol-2-yl-piperidin-4-ylamino]-1-(3,5-difluoro-benzyl)-2-hydroxy-propyl]-acetamide.

This invention is also directed to:

pharmaceutical compositions containing such beta-secretase inhibitors of formula I above or a pharmaceutically acceptable salt thereof and a pharmaceutically acceptable carrier for use in the methods disclosed herein;

a method of treatment of a disorder or condition that may be treated by inhibiting beta-secretase, the method comprising administering to a mammal in need of such treatment a therapeutically effective amount of a compound of formula I or a pharmaceutically acceptable salt thereof;

a pharmaceutical composition for treating a CNS condition, for example, a disorder or condition selected from the group consisting of Alzheimer's disease (AD), mild cognitive impairment, Down's syndrome, Hereditary Cerebral Hemorrhage with Amyloidosis of the Dutch-Type, cerebral amyloid angiopathy, dementias of mixed vascular and degenerative origin, dementia associated with Parkinson's disease, dementia associated with progressive supranuclear palsy, dementia associated with cortical basal degeneration, multi-infarct dementia, alcoholic dementia or other drug-related dementia, dementia associated with intracranial tumors or cerebral trauma, dementia associated with Huntington's disease, or AIDS-related dementia, diffuse Lewy body type of Alzheimer's disease, frontotemporal dementias with parkinsonism (FTDP), inclusion body myocytes, supranuclear cataracts, age-related macular degeneration (AMD), Huntington's disease, Parkinson's Disease, Restless Leg Syndrome, stroke, head trauma, spinal cord injury, demyelinating diseases of the nervous system, peripheral neuropathy, pin, cerebral amyloid angiopathy, amyotrophic lateral sclerosis, multiple sclerosis, dyskinesia associated with dopamine agonist therapy, mental retardation, learning disorders, including reading disorder, mathematics disorder, or a disorder of written expression; age-related cognitive decline, amnesic disorders, neuroleptic-induced parkinsonism, tardive dyskinesias, Tourette's syndrome, Multiple Sclerosis, and acute and chronic neurodegenerative disorders, the composition comprising a compound of formula I or a pharmaceutically acceptable salt thereof and a pharmaceutically acceptable carrier;

a method of treatment of a disorder or condition selected from the group consisting of the disorders or conditions listed herein, the method comprising administering to a mammal in need of such treatment a compound of formula I or a pharmaceutically acceptable salt thereof;

a pharmaceutical composition for preventing or delaying the onset of AD, preventing or delaying the onset of AD in patients who would otherwise be expected to progress from mild cognitive impairment (MCI) to AD, or preventing potential consequences of cerebral amyloid angiopathy such as single and recurrent lobar hemorrhages, the composition comprising a compound of formula I or a pharmaceutically acceptable salt thereof and a pharmaceutically acceptable carrier; and

a method for preventing or delaying the onset of AD, preventing or delaying the onset of AD in patients who would otherwise be expected to progress from MCI to AD, or preventing potential consequences of cerebral amyloid angiopathy such as single and recurrent lobar hemorrhages, the method comprising administering to a patient in need of such treatment a compound of formula I or a pharmaceutically acceptable salt thereof.

The invention also provides the use of a compound or salt according to formula I for the manufacture of a medicament.

The invention also provides compounds, pharmaceutical compositions, kits, and methods for inhibiting beta-secretase-mediated cleavage of amyloid precursor protein (APP), protein, the method comprising administering to a patient in need of such treatment a compound of formula I or a pharmaceutically acceptable salt thereof. More particularly, the compounds, compositions, and methods of the invention are effective to inhibit the production of A-beta and to treat or prevent any human or veterinary disease or condition associated with a pathological form of A-beta.

The compound of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

Isotopically-labeled compounds of formula I or pharmaceutically acceptable salts, thereof, including compounds of formula I isotopically-labeled to be detectable by PET or SPECT, are also within the scope of the invention.

Cis and trans isomers of the compound of formula I or a pharmaceutically acceptable salt thereof are also within the scope of the invention.

Tautomers of the compound of formula I or a pharmaceutically acceptable salt thereof are also within the scope of the invention.

When a first group or substituent is substituted by two or more groups or substituents, the invention includes without limitation embodiments in which a combination of such groups or substituents is present.

When a first group or substituent is substituted by two or more groups or substituents, it is understood that the number of such substituents may not exceed the number of positions in the first group or substituent that are available for substitution.

“Halogen” and “halo” and the like independently includes fluoro (F), chloro (Cl), bromo (Br) and iodo (I).

“Alkyl” including as may appear in the terms “alkoxy,” “thioalkoxy” and “alkyoxy” and the like includes saturated monovalent hydrocarbon radicals having straight or branched moieties. Examples of alkyl groups include, but are not limited to, methyl, ethyl, n-propyl, isopropyl, and t-butyl.

“Alkenyl” and “Alkynyl” include alkyl moieties having at least one carbon-carbon double or triple bond, respectively.

“Cycloalkyl” includes non-aromatic saturated cyclic alkyl moieties wherein alkyl is defined as above. Examples included without limitation: cyclopropyl, cyclobutyl, cyclopentyl, cyclohexyl, cycloheptyl; and bicycloalkyl and tricycloalkyl groups that are non-aromatic saturated carbocyclic groups consisting of two or three rings respectively wherein said rings share at least one carbon atom. Unless otherwise indicated herein bicycloalkyl groups include spiro groups and fused ring groups, e.g. bicycle-[3.1.0]-hexyl, bicycle-[2.2.1]-hept-1-yl, norbornyl, spiro[4.5]decyl, spiro[4.4]nonyl, spiro[4.3]octyl and spiro[4.2]heptyl. An example of a tricycloalkyl group is adamantanyl. Cycloalkyl groups also include groups substituted with one or more oxo moieties, e.g. oxocyclopentyl and oxocyclobutyl.

As appreciated, the term (CH₂)₀₋₅ and the like denotes the optional presence of a methylene linkage up to the carbon number indicated (here, 5), the connecting substituent to which may be in the normal or branched configuration, e.g. in (CH₂)₀₋₅(C₆₋₁₀aryl) the aryl may be in the branched or normal position in the methylene chain.

The term “alkyl”, “alkoxy”, “thioalkoxy”, “alkyoxy”, “alkenyl”, “alkynyl”, “cycloalkyl” as defined and used herein are further intended to include moieties of same that may each be optionally substituted with up to 3 fluoros (F) irrespective of whether such substitutions are; specifically mentioned as optional or otherwise.

“Treatment” and “treating” refers to reversing, alleviating, inhibiting the progress of, or preventing the disorder or condition to which such term applies, or one or more symptoms of such condition or disorder. As used herein, the term also encompasses, depending on the condition of the patient, preventing the disorder, including preventing onset and/or recurrence of any symptoms associated therewith, as well as reducing the severity of the disorder or any of its symptoms prior to onset.

“Mammal” refers to any member of the class “Mammalia”, including, but not limited to, humans, dogs, and cats.

“patient” refers to a member of the class Mammalia, including humans.

“Condition” refers to a disease or disorder.

“Heteroaryl” refers to a heteroaryl group constituted of one or more aromatic groups containing one or more heteroatoms (O, S, or N), preferably from one to four heteroatoms. As used herein, a multicyclic group containing one or more heteroatoms wherein at least one ring of the group is aromatic is also a “heteroaryl” group. The heteroaryl groups of this invention can also include ring systems which exist in one or more tautomeric forms (e.g. keto, enol, and like forms), and/or substituted with one or more oxo moieties. Examples of heteroaryl groups are, without limitation: quinolyl, isoquinolyl, 1,2,3,4-tetrahydroquinolyl, 1,2,4-triazinyl, 1,3,5-triazinyl, 1-oxoisoindolyl, furazanyl, benzofurazanyl, benzothiophenyl, dihydroquinolyl, dihydroisoquinolyl, benzofuryl, furopyridinyl, pyrolopyrimidinyl, and azaindolyl, pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocumarinyl, chromonyl, chromanonyl, pyridinyl-N-oxide, tetrahydroquinolinyl, dihydroquinolinyl, dihydroquinolinonyl, dihydroisoquinolinonyl, dihydrocumarinyl, dihydroisocumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide

“Heterocycloalkyl” and “Heterocylic” refer to a heterocycloalkyl group of one or more non-aromatic cyclic groups containing one or more heteroatoms, preferably from one to four heteroatoms, each selected from O, S and N. Heterocyclic groups also include ring systems substituted with one or more oxo moieties. Without limitation, examples of heterocyclic groups include: aziridinyl, azetidinyl, azepinyl, 1,2,3,6-tetrahydropyridinyl, oxiranyl, oxetanyl, tetrahydrothiopyranyl, morpholino, thiomorpholino, indolinyl, 2H-pyranyl, 4H-pyranyl, dioxanyl, 1,3-dioxolanyl, pyrazolinyl, dihydrothienyl, dihydrofuranyl, pyrazolidinyl, imidazolinyl, imidazolidinyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl, morpholinyl, thiomorpholinyl, thiomorpholinyl 8-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide.

The foregoing groups, as derived from the compounds listed above, may be C-attached or N-attached where such is possible. For instance, a group derived from pyrrole may be pyrrol-1-yl (N-attached) or pyrrol-3-yl (C-attached). The terms referring to the groups also encompass all possible tautomers.

As used herein: Ac=acetyl; BOC=t-butoxycarbonyl; EDC=1,(3, dimethylaminopropyl)-3-ethyl-carbodiimide hydrochloride; CBZ=benzyloxycarbonyl; THF=tetrahydrofuran; DPPP=1,3-bis(diphenylphosphinyl)propane; dba=dibenzylideneacetone; Et=ethyl; Me=methyl; n-Bu=n-butyl; n-Hex=n-hexyl; DMF=dimethylformamide, DCM=dichloromethane, CHCL3=chloroform, CDCl3=deuterochloroform, TFA=trifluoroacetic add, ES=Electrospray, LC=liquid chromatography, HPLC=high pressure liquid chromatography, MS=mass spectrometry, CD3OD=deuteromethanol, FMOC=fluorenylmethyloxycarbonyl, nBuLi=n-butyl lithium, MeOH=methyl alcohol, DIEA=diisopropylethylamine, LCMS=liquid chromatography mass spectrometry and MsCl=methane sulfonyl chloride.

CNS conditions subject of the invention are those known in the art; and include without limitation those wherein an inhibitor to beta-secretase is indicated.

The compound of the invention can also be used in combination with other drugs, e.g. those conventionally used to treat any of the CNS conditions herein described. For example, the compound of the invention can be used in combination with donepezil and like compounds to treat neurodegenerative diseases such as Alzheimer's Disease; or with selective serotonin reuptake inhibitors (SSRIs) and like compounds to treat depression.

In practice, the IC₅₀ value of the compounds of the invention in a BACE assay as described herein is in the range of from about 1 nanomolar to about 10 micromolar.

Cell Free BACE1 Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds. The substrate is Biotin-GLTNIKTEEISEISŶEVEFR-C[oregon green]KK-OH. The enzyme (0.1 nanomolar) and test compounds (0.00002-200 micromolar) are incubated in pre-blocked, low affinity, black plates (384 well) at RT for 30 minutes. The reaction is initiated by addition of 150 millimolar substrate to a final volume of 30 microliter per well. The final assay conditions are: 0.00002-200 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin (0.75 micromolar). After incubation with streptavidin at room temperature for 15 minutes, fluorescence polarization is measured, for example, using a PerkinElmer Envision (Ex485 nm/Em530 nm). The activity of the beta-secretase enzyme is detected by changes in the fluorescence polarization that occur when the substrate is cleaved by the enzyme. Incubation in the presence of compound inhibitor demonstrates specific inhibition of beta-secretase enzymatic cleavage of its synthetic APP substrate.

The ensuing methods and examples illustrate, without limitation, representative ways to make the compound of the invention.

Dosage Forms and Amounts

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration: and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form Is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

Given a particular compound of the invention and a desired dosage form, one skilled in the art would know how to prepare and administer the appropriate dosage form:

The compounds of the invention are used in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

In addition, the compounds of formula (I) can also be used with inhibitors of P-glycoprotein (P-gp). P-gp inhibitors and the use of such compounds are known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of formula (A). To that end the P-gp inhibitor and the compounds of formula (A) can be administered at the same time, by the same or different route of administration, orr at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It is to be understood that additional agents will be found that have the same function and therefore achieve the same outcome; such compounds are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a patient may be started on one dose, that dose may have to be varied over time as the patient's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the patient one thru four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ.

The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the patch is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art.

The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

It should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in this art.

Inhibition of APP Cleavage

The compounds of the invention generally inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in PCT patent applications WO01/23533, and WO00/17369, as well as in literature publications (Hussain et al., 1999, Mol. Cell. Nourosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460). Synthetic forms of the enzyme have also been described (WO00/17369). Beta-secretase can be extracted and purified from human brain tissue and can be produced in cells, for example mammalian cells expressing recombinant enzyme.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:214. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-W. The MBP portion is captured on an assay substrate by anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

In Vivo Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a patient at risk for the disease.

Unless defined otherwise, all scientific and technical terms used herein have the same meaning as commonly understood by one of skill in the art to which this invention belongs. All patents and publications referred to herein are hereby incorporated by reference for all purposes.

All patents and publications referred to herein are hereby incorporated by reference for all purposes.

Structures were named using Name Pro IUPAC Naming Software, version 5.09, available from Advanced Chemical Development, Inc., 90 Adelaide Street West, Toronto, Ontario, M5H 3V9, Canada.

The following examples are intended to be representative of specific embodiments of the invention, and are not intended as limiting the scope of the invention.

### BIOLOGY EXAMPLES

### Example A

### Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds of the invention. Useful substrates include the following:

Biotin-SEVNL-DAEFRC[oregon green]KK[SEQ ID NO: 1]

Biotin-SEVKM-DAEFRC[oregon green]KK[SEQ ID NO: 2]

Biotin-GLNIKTEEISEISY-EVEFRC[oregon green]KK[SEQ ID NO: 3]

Biotin-ADRGLTTRPGSGLTNIKTEEISEVNL-DAEFRC

[oregongreen]KK[SEQ ID NO:4]

Biotin-FVNQHLCoxGSHLVEALY-LVCoxGERGFFYTPKAC

[oregon green]KK[SEQ ID NO: 5]

The enzyme (0.1 nanomolar) and test compounds (0.001-100 micromolar) are incubated in pre-blocked, low affinity, black plates (384 well) at 37° C. for 30 minutes. The reaction is initiated by addition of 150 millimolar substrate to a final volume of 30 microliter per well. The final assay conditions are: 0.001-100 micromolar compound inhibitor, 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37° C., and the reaction is terminated by the addition of a saturating concentration of immunopure streptavidin. After incubation with streptavidin at room temperature for 15 minutes, fluorescence polarization is measured, for example, using a LJL Acqurest (Ex485 nm/Em530 nm). The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate. In this assay, preferred compounds of the invention exhibit an IC₅₀ of less than 50 micromolar. More preferred compounds of the invention exhibit an IC₅₀ of less than 10 micromolar. Even more preferred compounds of the invention exhibit an IC₅₀ of less than 5 micromolar.

### Example B

### Beta-Secretase Inhibition

### P26-P4′SW Assay

Synthetic substrates containing the beta-secretase cleavage site of APP are used to assay beta-secretase activity, using the methods described, for example, in published PCT application WO00/47618. The P26-P4′SW substrate is a peptide of the sequence:

(biotin)CGGADRGLTTRPGSGLTNIKTEEISEVNLDAEF [SEQ ID NO: 6]

The P26-P1 standard has the sequence:

(biotin)CGGADRGLTTRPGSGLTNIKTEEISEVNL [SEQ ID NO: 7].

Briefly, the biotin-coupled synthetic substrates are incubated at a concentration of from about 0 to about 200 micromolar in this assay. When testing inhibitory compounds, a substrate concentration of about 1.0 micromolar is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 picomolar, after dilution.

The reaction mixture also includes 20 millimolar sodium acetate, pH 4.5, 0.06% Triton X100, and is incubated at 37° C. for about 1 to 3 hours. Samples are then diluted in assay buffer (for example, 145.4 nanomolar sodium chloride, 9.51 millimolar sodium phosphate, 7.7 millimolar sodium azide, 0.05% Triton X405, 6 g/liter bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 hours at 4° C. After washing in TTBS buffer (150 millimolar sodium chloride, 25 millimolar Tris, 0.05% Tween 20, pH 7.5), the samples are incubated with streptavidin-AP according to the manufacturer's instructions. After a one hour incubation at room temperature, the samples are washed in TTBS and incubated with fluorescent substrate solution A (31.2 g/liter 2-amino-2-methyl-1-propanol, 30 mg/liter, pH 9.5). Reaction with streptavidin-alkaline phosphate permits detection by fluorescence. Compounds that are effective inhibitors of beta-secretase activity demonstrate reduced cleavage of the substrate as compared to a control.

### Example C

### Assays Using Synthetic Oligopeptide-Substrates

Synthetic oligopeptides are prepared that incorporate the known cleavage site of beta-secretase, and optionally detectable tags, such as fluorescent or chromogenic moieties. Examples of such peptides, as well as their production and detection methods are described in U.S. Pat. No. 5,942,400, herein incorporated by reference. Cleavage products can be detected using high performance liquid chromatography, or fluorescent or chromogenic detection methods appropriate to the peptide to be detected, according to methods well known in the art.

By way of example, one such peptide has the sequence SEVNL-DAEF [SEQ ID NO: 8], and the cleavage site is between residues 5 and 6. Another preferred substrate has the sequence ADRGLTTRPGSGLTNIKTEEISEVNL-DAEF [SEQ ID NO: 9], and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of the compound inhibitor to control results provides a measure of the compound's inhibitory activity.

### Example D

### Inhibition of Beta-Secretase Activity

### Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651 Met52 to Asn651 Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (diluted in DMSO) at the desired concentration, generally up to 10 micrograms/ml. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

### Example E

### Inhibitlon of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature 373:523-527 are useful to analyze in vivo suppression of A beta release in the presence of putative inhibitory compounds. As described in U.S. Pat. No. 6,191,166, 4 month old PDAPP mice are administered compound formulated in vehicle, such as corn oil. The mice are dosed with compound (1-30 mg/ml; preferably 1-10 mg/ml). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days. Beginning at time 0, brain tissue or cerebral fluid is obtained from selected animals and analyzed for the presence of APP cleavage peptides, including A beta, for example, by immunoassay using specific antibodies for A beta detection. At the end of the test period, animals are sacrificed and brain tissue or cerebral fluid is analyzed for the presence of A beta and/or beta-amyloid plaques. The tissue is also analyzed for necrosis.

Animals administered the compound inhibitors of the invention are expected to demonstrate reduced A beta in brain tissues or cerebral fluids and reduced beta amyloid plaques in brain tissue, as compared with non-treated controls.

### Example F

### Inhibition of A Beta Production in Human Patients

Patients suffering from Alzheimers Disease (AD) demonstrate an increased amount of A beta in the brain. AD patients are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated patients.

### Example G

### Prevention of A Beta Production in Patients at Risk for AD

Patients predisposed or at risk for developing AD are identified either by recognition of a familial inheritance pattern, for example, presence of the Swedish Mutation, and/or by monitoring diagnostic parameters. Patients identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated patients.

The invention has been described with reference to various specific and preferred embodiments and techniques. However, it should be understood that many variations and modifications may be made while remaining within the spirit and scope of the invention.

### DETAILED DESCRIPTION OF THE INVENTION

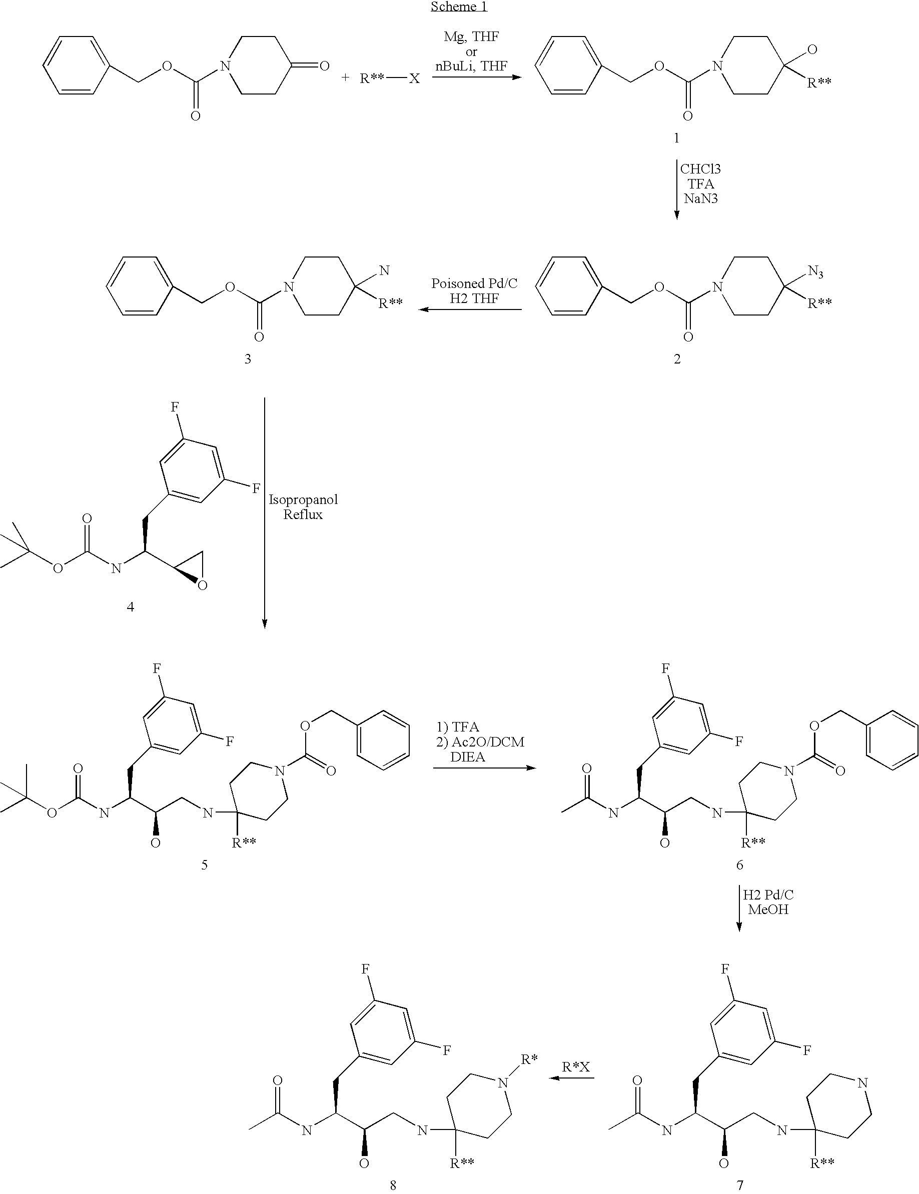

The synthesis of piperidine inhibitor 8 as illustrated in Scheme 1 starts with commercially available N-carbobenzyloxypiperid-4-one. A Grignard reagent of an aryl halide is formed by reaction with magnesium metal in ether and reacted with the ketone to give a tertiary carbinol 1. Alternatively, the aryl lithium reagent can be prepared from an aryl halide by metal halogen exchange and then added to a solution of N-carbobenzyloxypiperid-4-one to give 1. Carbinol 1 is converted to the azide 2 by reaction with sodium azide in a cold mixture of chloroform and trifluoroacetic acid. The azide is selectively reduced to the primary amine 3 by hydrogenation using the poisioned palladium catalyst described by H. Sajiki et. al. (J. Org. Chem. 1998, 63, 7990). Primary amine 3 is reacted with chiral epoxide 4 (CAS 388071-27-0 see Reeder, Michael R. WO 2002085877 A2) to provide the Boc and Cbz protected triamine 5. Compound 5 is tBoc deprotected by treatment with trifluoracetic acid. Evaporation of the solvent affords a TFA salt, which is selectively acetylated on the primary nitrogen using 1.0 equivalents of acetic anhydride in DCM in the presence of several equivalents of tertiary amine base to provide Cbz protected amine 6. The Cbz protecting of 6 group is removed by catalytic hydrogenation to provide key intermediate 7. Reaction of 7 with suitable electrophiles such as alkyl halides, aryl halides, carboxylic acids, carboxylic acid chlorides, active esters of carboxylic acids, sulfonyl chlorides, aldehydes by the process of reductive amination, ketones by the process of reductive amination, etc. to make a wide variety of compounds generically represented by 8.

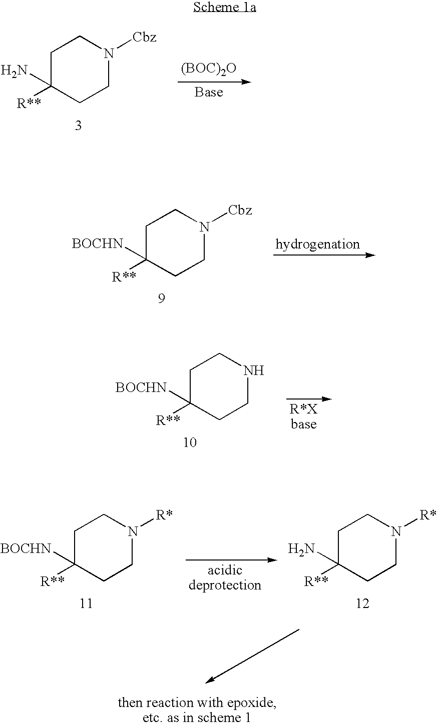

Scheme 1a details an alternate general procedure for preparing the substituted piperidine compounds (8) of Scheme 1 when late stage derivatization of piperidine intermediate 7 is not feasible as in Scheme 1. Thus the primary amine of compound 3 is protected with a BOC group to yield compound 9, then the piperidine nitrogen is deprotected by hydrogenation with palladium on carbon in ethanol to yield compound 10 which may be derivatized by methods described in scheme 1 or by other standard methods known to those skilled in the art to give compound 12 which is then carried to the compound of type 8 as described in Scheme 1.

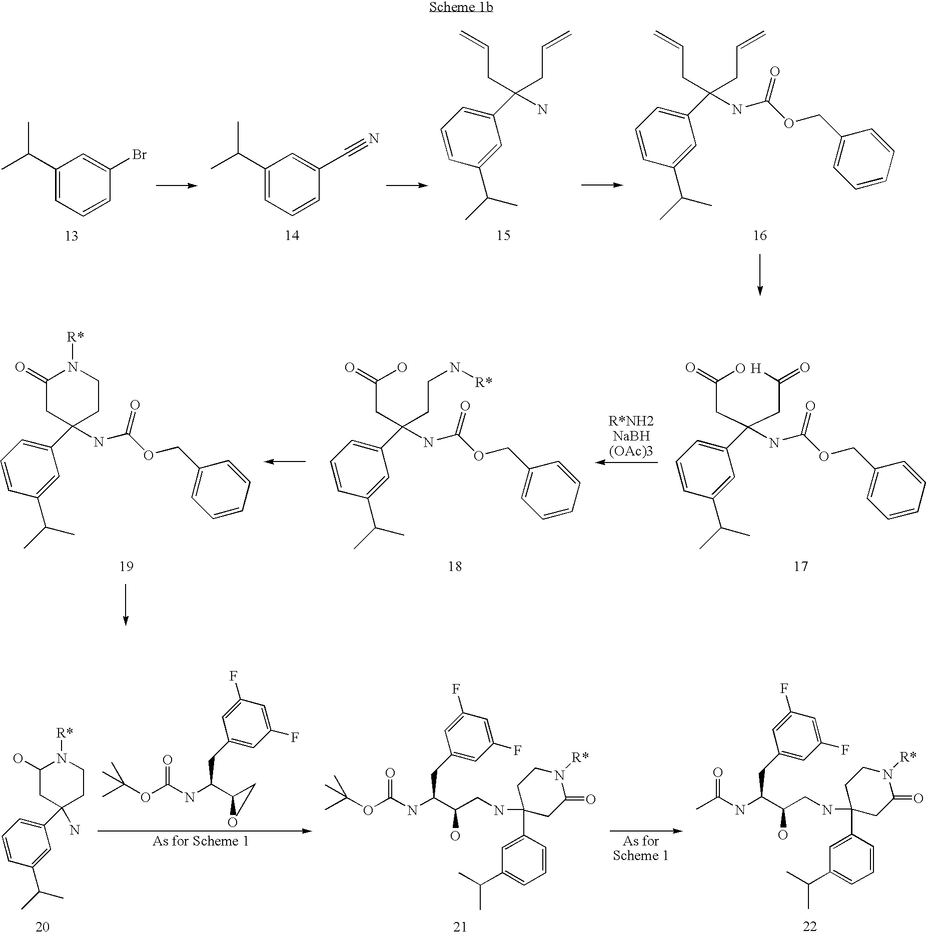

Scheme 1b details the synthesis of piperidone inhibitors 22. The starting material was m-isopropylbenzonitrile 14, which was synthesized from the commercially available 3-isopropylbromobenzene 13 by the procedure of Newman and Easterbrook (Am. Chem. Soc. 77, 1955, 3763). Nitrile 14 underwent double addition of allyl nucleophile using the organosamarium reagent described by Yu and Zhang (Syn. Comm. (1997), 27(9), 1495) to give primary amine 15. The amine can be purified at this time or it can be protected with a Cbz group using benzylchloroformate under standard conditions to give intermediate 16, which is then purified by silica gel chromatography. Oxidation of the terminal olefins of 16 by treatment with ozone in methylene chloride and acetic acid at −78° C. gives a mixture of dialdehyde, aldehyde-acid 17 and diacid 28. Diacid 28 can be used in the synthesis of glutarimide based compounds such as 29-31 (see scheme 1d). In general, the aldehyde acid 17 is the major product and it is isolated by preparative reverse phase chromatography using a C18 column. An alternate and preferred synthesis of the methyl ester of 17 is described in Scheme 1c. Aldehyde-acid 17 readily undergoes reductive amination with primary amines to give the secondary amine-acid 18. Using LCMS to monitor the reactions, it was noticed that during reaction work-up, the amine-acid 18 can cyclize spontaneously to piperidone 19. In practice, the work-up product will contain both cyclic and products so it best to drive as much of the product to piperidone before purification. This can be done by refluxing for 15-30 min in a solvent such as CHCl3. Purified piperidone 19 was deprotected by catalytic hydrogenation to primary amine 20, which is reacted directly with chiral epoxide 4 to give the Boc protected inhibitor 21 as a 1:1 mixture of diasteromers. The Boc group is removed and the primary amine acetylated as in Scheme 1 to give inhibitors 22, which are 1:1 mixtures of diastereomers at the piperidine 4-position.

The diastereomers can be resolved using techniques well known in the art including, Chromatography. Examples include preparative reverse phase chromatography on a C18 column or regular phase chromatography on a silica gel column. In general, one diastereomer is more active than the other. It is well known in pharmaceutical science that differing stereoisomers can have differing biological activities. These activities can be determined by separating the stereoisomers using a variety of well known resolving techniques and testing the stereoisomers as separate entities. The absolute stereochemistry at each site within an active stereoisomer can be determined by a variety of methods including X-ray crystallography or stereoselective synthesis. These methods can be performed by those skilled in the art. The present invention includes mixtures of diastereomers and the resolved, homochiral species. Resolved single enantiomer isomers are preferred embodiments of the present invention. When one resolved stereoisomer is found to have greater potency against BACE than the other, then that isomer is especially preferred.

Resolved diastereomers are included in the examples (see Example 2). As expected, once resolved the two diastereomers exhibit differing abilities to inhibit the BACE enzyme. In one instance, the absolute stereochemistry of the more potent diastereomer has been determined by crystallography and found to have the IUPAC S configuration. It is reasonable to conclude that the same absolute stereochemistry will have higher potency throughout the piperidone series and thus these diastereomers are preferred embodiments.

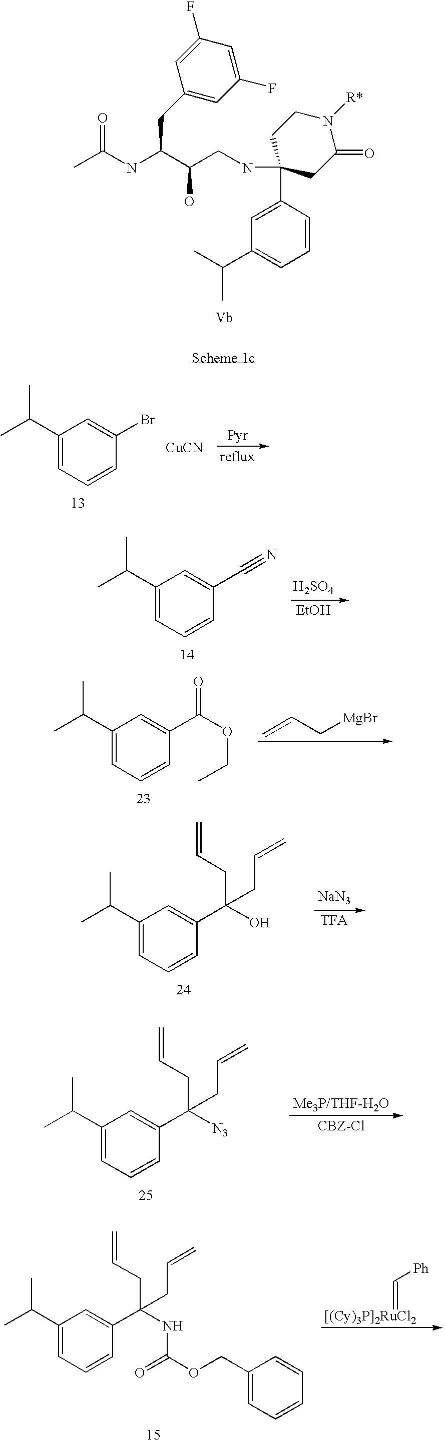

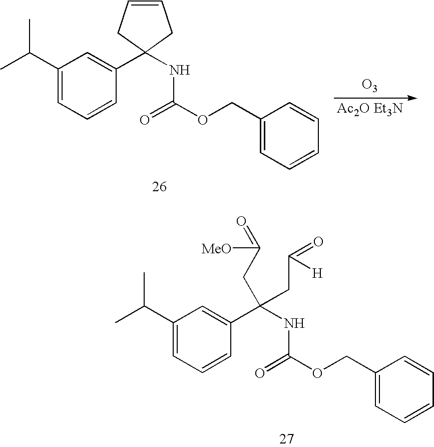

Scheme 1c outlines an alternate and preferred synthesis of compound 22 (scheme 1b). In this procedure, m-isopropylbenzonitrile was converted into an alkyl ester 23 including but not limited to the ethyl ester by dissolution in an alcohol such as ethanol followed by treatment with an acid including but not limited to hydrochloric, sulfuric, phosphoric, methanesulfonic, trifluoroacetic or trifluoromethanesulfonic acid. The preferred acid is sulfuric. The reaction may be run at room temperature up to the reflux point of the solvent. The preferred conditions include running the reaction in refluxing ethanol. The resulting ester is treated with an allylic organometallic in an ether solvent at a temperature between −100° C. and the reflux point of the solvent. Typical allylic organometallics include allyl magnesium bromide, allyl magnesium iodide, allyl magnesium chloride, or allyl lithium with allyl magnesium bromide being preferred. Suitable solvents include diethyl ether, THF, dioxane with THF being preferred. The reaction is preferably run at room temperature but may also be run at reflux in THF. The resulting tertiary alcohol 24 is treated with an azide salt in the presence of an acid in order to form a tertiary azide 25. Suitable azide salts include sodium, potassium, lithium or tetrabutyl ammonium azide as well as other sources of azide ion. The most preferred is sodium azide. Typical acids include trifluoroacetic acid, acetic acid, triflic acid, methanesulfonic acid, hydrochloric acid, sulfuric acid or other similar acids. The most preferred is trifluoroacetic acid. The reaction is run in an inert solvent such as methylene chloride, chloroform, dichloroethane or the like. The most preferred solvent is chloroform. The reaction is typically run below room temperature and is preferably run at −20 to −10° C. The tertiary azide can be reduced by a variety of methods. Suitable conditions include hydrogenation at from 1-50 atm of hydrogen pressure using heterogenous catalysts such as derived from palladium, platinum or nickel for instance Raney nickel. The reaction can be run in a variety of inert solvents including alcohols such as methanol or ethanol or inert solvents such as ethyl acetate or THF. The reaction can be run in acidic solvents such as acetic acid or in the presence of acetic acid in combination with any of the previous solvents. The hydrogenation is typically run at room temperature or below room temperature. The tertiary azide may also be reduced through reaction with phosphines or sulfides. For instance reaction of the azide with triphenylphosphine or trimethylphosphine in a suitable inert solvent containing water under the well known Staudinger reaction conditions is followed by hydrolysis to the requisite amine, phosphine oxide and nitrogen gas in situ. This reaction may be run at room temperature up to the reflux point of the solvent. The most preferred conditions include reaction of the azide in THF containing water with trimethyl phosphine at room temperature. The resulting amine can be protected with a variety of urethane based protecting groups including: t-BOC, FMOC, TrOC or CBz. The most preferred group, Cbz may be introduced from CBz-chloride in a variety of solvents or combination of solvents to generate protected amine 16. When water is used alone or in combination with other inert solvents, it usually contains an inorganic base such as sodium or potassium carbonate or bicarbonate or sodium or potassium hydroxide. When inert organic solvents are used alone then usually they contain a tertiary amine base such as triethylamine or diisopropylethylamine. Suitable inert organic solvents include dichloromethane, dichloroethane, chloroform, toluene, or dioxane. The preferred conditions include CBz-Cl in a mixture of dioxano-water using sodium bicarbonate as base. The reaction is optimally run at room temperature but can be run at 50° C. to the reflux point of the solvent mixture. Ring closing metathesis of the diolefin to cyclic olefin 26 is carried out under the conditions pioneered by Grubbs (Accounts of Chemical Research 28 (1995) 446-452) using his first generation ruthenium catalysts as depicted in the following references. S. Kotha et al Biorg and Medicinal Chemistry Letters 11 (2001) 1421-1423, S. Kotha et al Biorg and Medicinal Chemistry Letters 8 (1998) 257-260, K. Unheim Tetrahedron 53 (1997) 2309-2322. The selective olefin ozonolysis with differentiation of the oxidized carbons to afford 27 was conducted under the procedure of S. Schreiber et al Tetrahedron Letters 23 (1982) 3867-3870. Compound 27 can be substituted for compound 17 and can be used directly as depicted in scheme 1b.

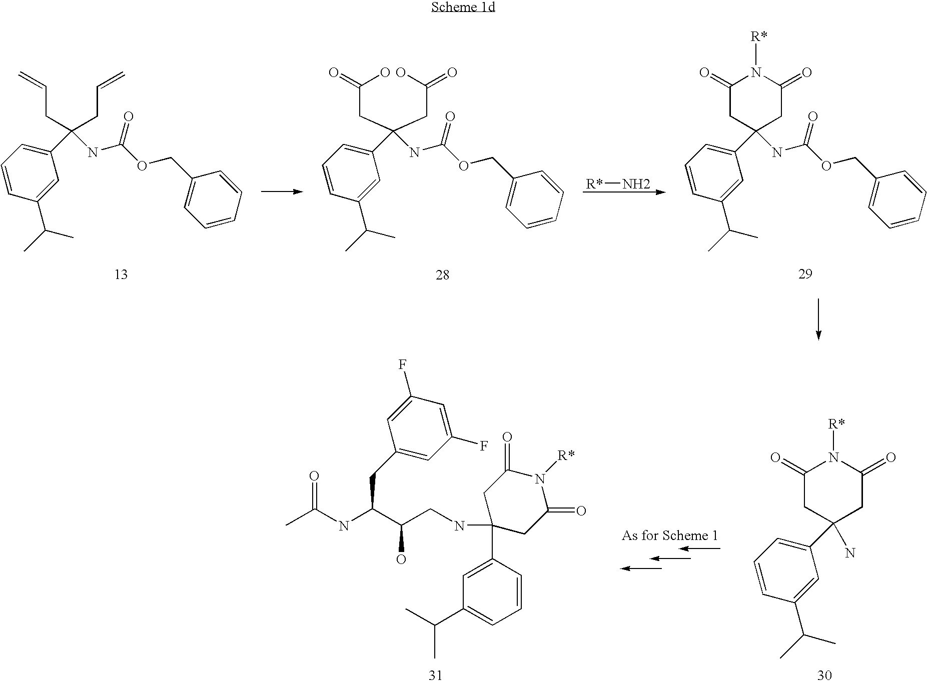

The synthesis of glutarimide based inhibitors, illustrated in Scheme 1d, starts with diacid 28, which is a side product created during the ozonolysis of diene 16 (see Scheme 1a). Diacid 28 can be recovered during the purification of acid aldehyde 17 as described in in the examples. Reaction of 28 with primary amines under dehydration conditions affords glutarimide 29. Catalytic hydrogenation of 29 provides primary amine 30, which can be reacted with chiral epoxide 4 as described in Scheme 1. Deprotection of the N-terminal Boc group followed by acetylation gives rise to inhibitor compound 31.

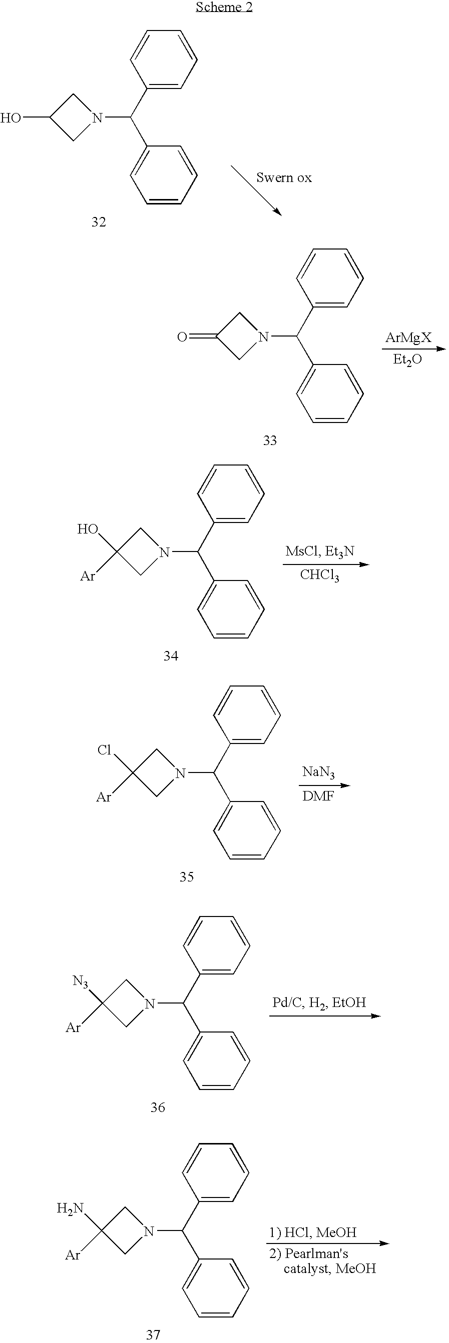

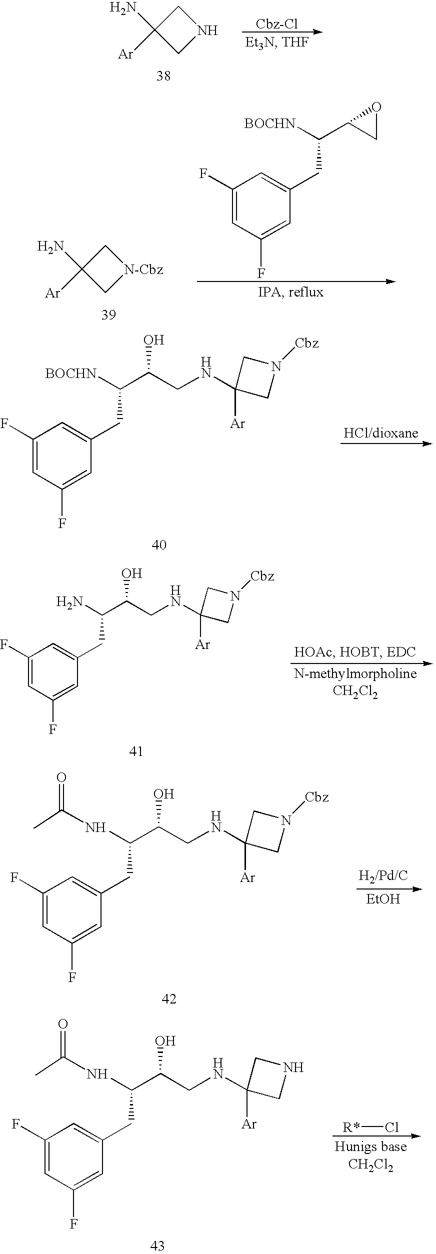

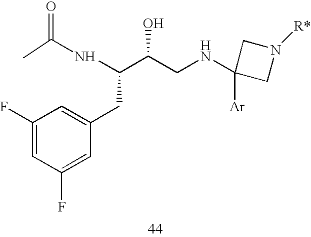

Azetidine structures of the type 44 may be prepared as shown on Scheme 2. Azetidinone 33 may be prepared from azetidinol 32 by oxidation with reagents such as Dess-Martin periodinane, sulfur trioxide-pyridine complex or preferably by Swern oxidation. Addition of an aryl lithium species or preferably an aryl Grignard to 23 in a suitably inert solvent such as diethyl ether or THF provides carbinol 34. Following the general procedure of Bacque et al (Syn. Comm, 25(6), 803-812 (1995)), 34 is converted into chloride 35 by treatment with methanesulfonyl chloride in a halogenated solvent such as 1,2-dichloroethane, methylene chloride or preferably chloroform in the presence of a non-nucleophilic organic base such as Hunig's base or triethylamine, then subsequently to azide 36 via treatment with sodium azide in a polar non-reactive solvent where DMF is preferred. Hydrogenation using a palladium catalyst such as palladium hydroxide or preferably palladium on carbon in an alcoholic solvent such as methanol or preferably ethanol affords primary amine 37. Removal of the benzylhydryl amine protecting group to prepare diamine 33 is accomplished by first preparing an acidic salt of amine 37 with HBr, or preferably HCl and then hydrogenating with palladium hydroxide in an alcohol such as ethanol or preferably methanol. Selective re-protection of the secondary amine with the more labile carboxybenzyl (Cbz) group to afford amine 39 is accomplished by treatment of amine 38 with carboxybenzyl anhydride or preferably benzyl chloroformate in an inert solvent such as ether, methylene chloride or preferably THF in the presence of non-nucleophilic amine such as Hunig's base or triethylamine. Compound 40 may be obtained by combining epoxide 4 and amine 39 either neat or in an alcoholic solvent such as methanol, ethanol, t-butanol or preferably isopropanol at temperatures ranging from 50° C. to 150° C. where 70-130° C. is preferred. Removal of the BOC protecting group through the treatment of 35 with acids such as TFA or preferably HCl in unreactive solvents such as methylene chloride or 1,4-dioxane affords amine 41. Acetamide 42 is obtained from amine 41 by acetylation conditions such as treatment with acetic anhydride, or carbonyl diimidazole or by treatment with acetic acid in the presence of an amide coupling agent such as BOP, HBTU/HOBT or preferably EDC/HOBT in the presence of Hunig's base or triethylamine in a suitable inert solvent such as THF, dioxane or preferably methylene chloride. Compound 42 is then hydrogenated in the presence of a suitable catalyst where palladium on carbon is preferred in an alcoholic solvent such as methanol or preferably ethanol to afford amine 43 which may then be converted to compound 44 by treating with acid chlorides or isocyanates in the presence of a non-nucleophilic base such as triethyl amine or Hunig's base.

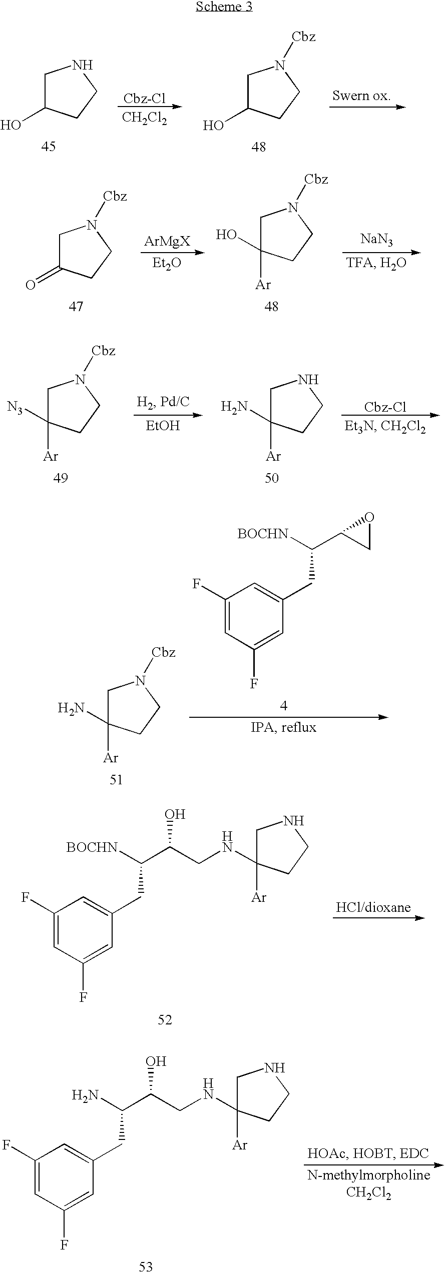

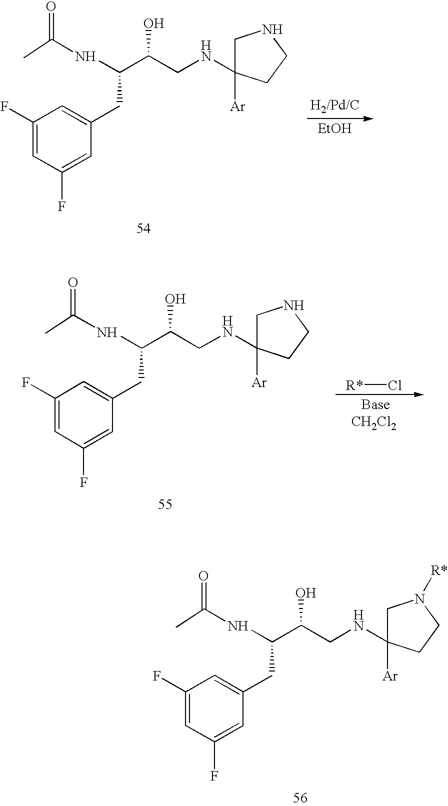

Scheme 3 describes the synthesis of pyrrolidine compounds (56). Protected pyrrolidinone 47 may be prepared by treatment of 3-hydroxypyrrolidine 45 with benzyl chloroformate In a suitably unreactive solvent such as THF, chloroform or preferably methylenechloride to afford compound 46 which may then be converted to compound 47 by oxidation with agents such as PCC, Dess-Martin periodinane or preferably using Swern oxidation conditions. Addition of an aryl lithium species or preferably an aryl Grignard to 47 in a suitably inert solvent such as diethyl ether or THF provides carbinol 48. Preparation of diamine 50 is achieved by first converting compound 48 to azide 49 using sodium azide and trifluoroacetic acid with no solvent or in suitable solvents such as chloroform, methylene chloride or preferably water at temperatures ranging from −10° C. to 50° C. where 0° to 25° C. is preferred, then by hydrogenating in the presence of palladium on carbon in an alcoholic solvent such as methanol or preferably ethanol to yield compound 50. Selective re-protection of the secondary amine of compound 50 to afford amine 51 is accomplished by treatment of with carboxybenzyl anhydride or preferably carboxylbenzyl chloride in an inert solvent such as ether, methylene chloride or preferably THF in the presence of non-nucleophilic amine such as Hunig's base or triethylamine. Conversion of compound 51 into compound of type 56 may be accomplished using the general method that was described in Scheme 2 for the conversion of compound 39 into azetidine 44.

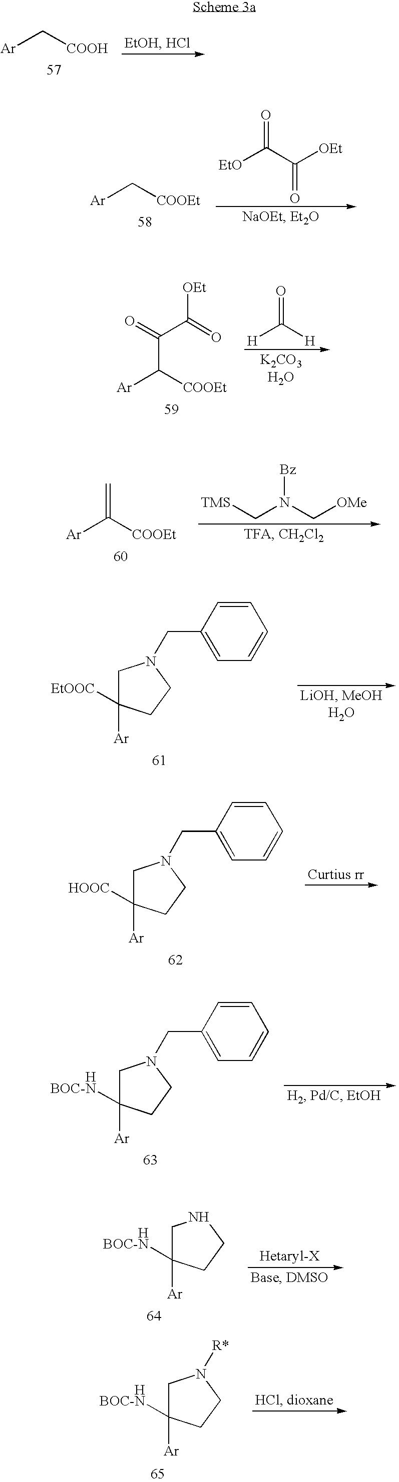

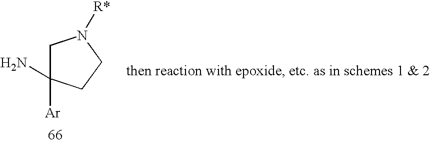

When derivatization of the pyrrolidine is not feasible as a final step as in scheme 3, the derivatized pyrrolidine amine may be prepared earlier in the sequence as described in Scheme 3a and then carried to compound 56 following the general method described in Schemes 1 and 2. Thus aryl acetic acid 57 is saponified to yield ester 58 and following the general procedure described by Jefford et al., Helv. Chim Acta, 69, 2048, 1986, converted to arylpropenoate 60 via treatment with ethyl glycolate and sodium methoxide in ether to form 59 and then with formaldehyde and aqueous K₂CO₃. Compound 60 is then treated with N-(methyloxymethyl)-N-(trimethylsilylmethyl)-benzyl amine and trifluoracetic acid in CH₂Cl₂ to yield N-benzyl pyrrolidine 61. Compound 61 may be saponified with alkali such as NaOH, KOH or preferably with LiOH in a mixture of water and an alcohol, where methanol is preferred to give acid 62 which is subjected to Curtius rearrangement conditions with diphenylphosphoryl azide and a non-nucleophile base such as Hunig's base or preferably triethylamine in t-butanol to afford the BOC protected amine 62. Deprotection of the pyrrolidine amine is achieved by hydrogenation with palladium on carbon in ethanol to yield compound 64. Derivatization of the pyrrolidine nitrogen of 859 is accomplished by methods described in scheme 2 or by other standard methods known to those skilled in the art to give compound 68 which is then carried on the compound of type 56 as described in schemes 1 and 2.

### Experimental

All reactions were run under a nitrogen atmosphere for convenience and to maximize yields. Melting points are uncorrected. Chromatography refers to flash chromatography on silica gel. NMR refers to proton [¹H] NMR. NMR spectra were obtained at 400 MHz and are reported in parts per million (δ) relative to the deuterium lock signal of the specified solvent. Evaporation or concentration implies the use of a rotary evaporation apparatus

### Example 1

### Synthesis of (2R,3S)-4-[3-Acetylamino-4-(3,5-difluoro-phenyl)-2-hydroxy-butylamino]-4-(3-isopropyl-phenyl)-piperidine-1-carboxylic acid methyl ester, trifluoracetic acid salt

### Step 1

### 4-Hydroxy-4-(3-Isopropyl-phenyl)-piperidine-1-carboxylic acid benzyl ester

In a dry 500 mL neck flask equipped with dropping funnel and reflux condenser and nitrogen inlet was placed 1.2 g Mg and a crystal of iodine. The flask was warmed to vaporize the iodine then 25 mL THF was added. A solution of 9.7 g (49 mmol) m-isopropylbromobenzene in 30 mL THF was added dropwise and the mixture refluxed until most of the Mg was consumed (about 1 hour). The resulting Grignard reagent was cooled to 5° C. and a solution of 10 g (43 mmol) N-Cbz-piperid-4-one (N-(carbobenzyloxy)-4-oxo-piperidine-1-carboxylic acid benzyl ester) in 50 mL THF was added dropwise over 15 mins. The reaction was allowed to warm to room temp for 2 hours then was quenched by slow addition of 150 mL sat. ammonium chloride solution. The mixture was diluted with 100 mL ether and the layers separated. The ether layer was washed again with saturated NaCl, dried and was evaporated. The oil was purified by silica gel chromatography (2:1 Hexane/EtOAc) and the product crystallized on standing. Yield=8.2 g (54%) Electrospray MS m/z=354.1 (MH+, expect 354.2) HNMR (CDCl3, 400 MHz) 7.2-7.4 ppm (m, 8H) 5.15 (s, 9H), 7.15 (d, 1H), 5.15 (s, 1H), 4.1 (bm, 2H), 3.3 (bm, 2H), 2.90 (m, 1H), 2.0 (bm, 2H), 1.75 (d, 2H), 1.25 (d, 6H, 8 Hz)

### Step 2

### 4-Azido4(3-isopropyl-phenyl)-piperidine-1-carboxylic acid benzyl ester

4.0 g of carbinol prepared in Step 1 was dissolved in 6 mL CHCl₃, 1.5 g NaN₃ was added and the mixture stirred at −5° C. in a salt ice bath. A solution of 12 ml TFA in 24 ml CHCl₃ was added to the mixture drop wise to the cold reaction mixture so that the reaction temperature did not exceed 0° C. (about 25 mins). After the addition, the ice bath was removed and the reaction was stirred at room temperature overnight. The reaction was diluted with 60 ml of EtOAc and washed with 2×60 ml water, and 1×50 ml brine. The EtOAc layer was evaporated and the crude product purified by silica gel chromatography (Hexanes with 0-10% EtOAc)

Yield=4.2 g (98%) Electrospray MS m/z=757.3 (M2H+, expect 757.4) 7.2-7.4 (m, 9H); 5.10 (s, 2H); 4.06 (dt, 2H); 3.25 (bs, □HNMR (CDCL3, 400 MHz) 2H); 2.88 (m, 1H); 1.98 (m, 4H), 1.23 (d, 6.5 Hz, 6H)

### Step 3

### 4-Amino-4-(3-isopropyl-phenyl)-piperidine-1-carboxylic acid benzyl ester

In 50 ml THF was dissolved 1.0 g of the compound prepared in Step 2. 200 mg of ethylenediamine poisoned 10% Pd/C was added (H. Sajiki et. al. J. Org. Chem. 1998, 63, 7990) and the mixture shaken under 50 psi hydrogen overnight. The reaction solution was filtered through Celite, evaporated and rotovap and high vacuum. Yield=0.98 g (105%). Electrospray MS m/z=705.4 (M2H+, expect 705.4)

### Step 4

### 4-[3-tort-Butoxycarbonylamino-4-(3,5-difluoro-phenyl)-2-hydroxy-butylamino]-4-(3-tert-butyl-phenyl)-piperidine-1-carboxylic acid benzyl ester

1.5 g (4.1 mmol) of the compound prepared in step 3 was dissolved in 3 ml 2-propanol and brought to gentle reflux. 1.3 g (4.3 mmol) of compound of formula 4 was added in aliquots every 30 min (4×200 mg, 3×100 mg, 4×50 mg) as this gives better yield than addition of a single aliquot. The reaction solution was diluted with 200 ml of EtOAc and washed with 2×200 ml 0.1 M NaHCO₃, and 1×50 ml brine. The EtOAc layer was evaporated and purified by silica gel chromatography using 50% EtOAc in hexane. Yield=1.4 g (51%) Electrospray MS: m/z=666.2 (MH+, expect 666.4)

### Step 5

### 4-(2R,3S)-[3-tert-Butoxycarbonylamino 4-(3,5-difluoro-phenyl)-2-hydroxy-butylamino]-4-(3-tert-butyl-phenyl)-piperidine-1-carboxylic acid benzyl ester

1.4 ml (2.1 mmol) of the compound prepared in Step 4 was dissolved in 5 ml neat TFA for 10 minutes then evaporated on rotovap followed by high vacuum. The TFA salt was dissolved in 50 ml DCM and 5 mL DIEA. 0.22 ml acetic anhydride was added, the solution stirred for 20 minutes then quenched with 1.0 mL methylamine in THF. The DCM was evaporated and the residue redissolved in 100 ml of EtOAc, washed with 2×100 ml water, 1×50 ml brine, then dried over MgSO4. Following solvent evaporation LCMS indicated that crude product was pure enough for use in the next step. Electrospray MS: m/z=608.3 (MH+, expect 608.3) HNMR (400 MHz, CD3OD) 7.43 (s, 1H); 7.17-7.35 (m, 8H); 6.77 (d, 2H); 6.68 (t, 1H): 5.15 (s, 2H), 4.07 (m, 1H): 3.75-3.5 (m, 4H); 3.45 (q, 1H); 3.00 (dd, 1H); 2.57 (dd, 1H); 2.24 (m, 2H); 1.8-2.0 (m, 4H); 1.73 (s, 3H); 1.29 (s, 9H)

### Step 6

### N-(1S,2R)-[3-[4-(3-tort-Butyl-phenyl)-piperidin-4-ylamino]-1-(3,5-difluoro-benzyl)-2-hydroxy-propyl]-acetamide

In 100 ml MeOH was dissolved 1.39 of the compound prepared in Step 5. 250 mg 10% Pd/C was added and the mixture shaken under 48 psi hydrogen overnight. The solution was passed through a pad of Celite and evaporated to give amine 7b in suitable purity for use in the next step. Yield from 1.4 g 5b=1.2 g (102%) Electrospray MS: m/z=474.2 (MH+, expect 474.2). HNMR (400 MHz, CD3OD) 7.46 (s, 1H); 7.25 (m, 3H); 6.74 (m, 3H); 4.04 (m, 1H); 3.45 (m, 1H); 3.14 (t, 2H); 2.98 (d, 3.3 Hz, 1H); 2.8 (m, 2H), 2.55 (dd, 14 Hz, 1H) 2.25 (m, 2H); 2.08 (m, 2H); 1.94 (m, 2H); 1.73 (s, 3H); 1.30 (s, 9H)

### Step 7

### (2R,3S)-4-[3-Acetylamino-4-(3,5-difluoro-phenyl)-2-hydroxy-butylamino]-4-(3-isopropyl-phenyl)-piperidine-1-carboxylic acid methyl ester, trifluoracetic acid salt

20 mg of the compound prepared in Step 6 was dissolved in 100 ul THF and 15 uL DIEA. 6.0 uL of methylchloroformate was added and the solution became a gel. 100 uL of THF was added and the mixture was vortexed at room temp for 10 min. The mixture was diluted with 0.5 mL methanol and the resulting solution was purified by direct injection into a prep HPLC system. Standard conditions for HPLC are as follows:

Prep column: BISCHOFF (Leonberg, Germany) C18, PREP2005, 20×50 mm.

Solvent A: 0.1% TFA, 5% ACN, H2O

Solvent B: ACN

Gradient: 0-80% B in 10 minutes

Flow rate: 10 ml/min,

Fraction size: 5 mL

Detection by UV absorbance@260 nm

Appropriate fractions were pooled and evaporated affording (2R,3S)-4-[3-Acetylamino-4-(3,5-difluoro-phenyl)-2-hydroxy-butylamino]-4-(3-isopropyl-phenyl)-piperidine-1-carboxylic acid methyl ester as a trifluoracetic acid salt.

Electrospray MS: m/z=518.22 (MH+, expect 518.3) HNMR (CD3OD) 7.67 (s, 1H); 7.56 (d, 1H); 7.48 (t, 9 Hz 1H); 7.41 (m, 1H); 6.77 (m, □ 3H); 3.85 (m, 1H); 3.75 (m, 2H); 3.67 (s, 3H); 3.58 (m, 1H); 3.20 (dd, 1H); 2.7-2.9 (m, 5H); 2.60 (dd, 1H); 2.55 (dd, 1H); 2.51 (m, 1H); 2.2-2.35 (dt 2H); 1.78 (s, 3H); 1.27 (d, 6.5 Hz, 6H)

In Examples 1a-1k listed below in Table 1, compounds represented by Formula IV were synthesized by steps similar to those described above for Example 1. These compounds were tested according to the BACE cell free assay described in Example B and exhibited IC₅₀ values in the range of from about 35 to about 7800 nanomolar.

TABLE 1
IV

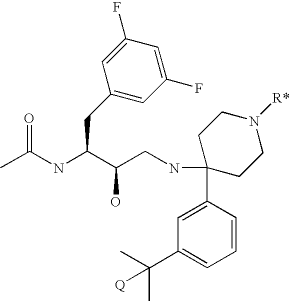

| | | | | ESMS MH+ |
|---|---|---|---|---|
| | | | | observed |
| Example | Q | R* | HNMR resonances ppm | (expected) |
| 1a | H | —COOCH₃ | 3.67 (s, 3H) | 518.18 (518.28) |
| 1b | H | —CO—CH₂OH | 4.20 (dq, 2H) | 518.22 (518.28) |
| 1c | H | —CO—COOCH₃ | 3.86 (s, 3H) | 546.21 (546.28) |
| 1d | H | —CO—COOH | — | 532.16 (532.26) |
| 1e | H | —SO₂—CH₂CF₃ | — | 606.12 (606.24) |
| 1f | H | —CO—NHCH₃ | 2.69 (s, 3H) | 517.29 (517.30) |
| 1g | H | —COOCH₂CH₃ | 2.65 (q, 2H); 1.27 (t, 3H) | 532.16 (532.30) |
| 1h | CH3 | —COOCH₃ | 3.67 (s, 3H) | 532.32 (532.30) |
| 1i | CH3 | —CO-nC₃H₇ | 2.41 (m, 2H), 1.05 (t, 3H) | 530.30 (530.30) |
| 1j | CH3 | —CON(CH₃)₂ | 2.90 (s, 6H) | 545.24 (545.27) |
| 1k | CH3 | —CO—CH₂CH(CH₃)₂ | 1.13 (d, 3H) 1.02 (t, 3H) | 544.36 (544.28) |

### Example 2

### Synthesis of N-{(1S,2R)-1-(3,5-Difluoro-benzyl)-3-[1-ethyl-4-(3-isopropyl phenyl)-2-oxo-piperidin-4-ylamino]-2-hydroxy-propyl}-acetamide

### Step 1

### 3-Amino-3-(3-isopropyl-phenyl)-penta-1,4-diene m-isopropylbenzonitrile

Using the procedure of Yu and Zhang (Syn. Comm. (1997), 27(9), 1495.) into a dry 500 mL three neck flask was placed 3.1 g of samarium metal (40 mesh) and 100 mL dry THF 2.9 g allyl bromide was added along with several small crystals of iodine. The mixture was stirred at room temperature. After 10 min the mixture turned to purple indicating the organosamarium reagent had formed. After 1 hr 1.34 g of m-isopropylbenzonitrile (synthesized by the procedure described by Newman and Easterbrook J. Am. Chem. Soc. 77, 1955, 3763) was added dropwise over 2 mins. The purple brown mixture was stirred for 2 h at room temp. 100 mL water was carefully added followed by 50 mL ether. The layers separated and the ether/THF layer was washed with sat. NaHCO3. The combined aqueous layers were again extracted with 50 mL ether and the combined organic layers were washed with brine, dried (MgSO4), and evaporated to a brown oil. The product was purified by flash silica gel chromatography (3:1 hexane/EtOAc). Yield=0.51 g. ESMS MH+=230.2. (MH-NH3)+=213.2. HNMR (CDCl3) 7.25 ppm (m, 3H), 7.07 (d, 1H, 7 Hz), 5.52 (m, 2H), 5.06 (d, 2H, 19 Hz), 5.02 (d, 2H, 8 Hz), 2.90 (p, 1H), 2.65 (dd, 2H), 2.42 (m, 2H), 1.23 (d, 6H, 6.6 Hz)

### Step 2

### [1-Allyl-1-(3 isopropyl-phenyl)-but-3-enyl]-carbamic acid benzyl ester

0.43 g of 3-Amino-3-(3-isopropyl-phenyl)-penta-1,4-diene was dissolved in 10 mL dioxane and 3 mL sat. Na2CO3. The solution was chilled to 5 C and 0.43 g of benxylchloroformate was added over 2 mins. The mixture was allowed to warn to room temp and was stirred well. After 30 min the reaction was diluted with 40 mL ether and 40 mL water. The layers separated and the ether layer was washed with 0.1M HCl, brine, dried (MgSO4), and evaporated. The product was purified by flash silica gel chromatography (5:1 hexane/EtOAc). Yield=0.52 g (77%). ESMS: MH+=364.2. HNMR (CDCl₃) 7.35 ppm (m, 5H), 7.22 (m, 1H), 7.11 (bs, 3H), 5.55 (m, 2H), 5.06 (d, 2H), 5.02 (4H), 2.97 (m, 2H), 2.93 (p, 1H), 2.65 (dd, 2H), 1.20 (d, 6H, 7 Hz)

### Step 3

### 3-Benzyloxycarbonylamino-3-(3-isopropyl-phenyl)-5-oxo-pentanoic acid and 3-Benzyloxycarbonylamino-3-(3-isopropyl-phenyl)-pentanedioic acid

0.62 g of [1-Allyl-1-(3-isopropyl-phenyl)-but-3-enyl]-carbamic acid benzyl ester was dissolved in 15 mL DCM and 1.5 mL AcOH. The solution was cooled in a dry ice acetone bath. Ozone was bubbled into the solution until a metallic blue color persisted. The reaction was stirred at −78 C for 20 mins then allowed to warm to room temperature. 7.5 mL AcOH was added and the DCM evaporated under stream of nitrogen. Another 7.5 mL AcOH was added along with 6.0 mL of 30% H2O2 in water. The reaction was refluxed for 2 hr. The reaction was evaporated, the residue dissolved in 30 mL CHCl3, filtered, evaporated. The crude products were purified by reverse phase HPLC as in Example 1 using a solvent gradient of, 20% acetonitrile->60% acetonitrile over 20 min.

Yield of compound 3-Benzyloxycarbonylamino-3-(3-isopropyl-phenyl)-5-oxo-pentanoic acid=0.15 g. ESMS MH+=384.4 (expect 384.3) HNMR (CDCl3) 9.60 ppm (s, 1H), 7.35 (m, 6H), 7.10 (m, 3H), 5.85 (bs, 1H), 5.05 (bs, 2H), 3.20 (m, 2H), 2.95 (m, 1H), 2.90 (m, 2H), 1.20 (d, 6H, 7 Hz).

Yield of compound 3-Benzyloxycarbonylamino-3-(3-isopropyl-phenyl)-pentanedioic acid=0.059 g. ESMS MNa+=422.2 (expect 422.3) HNMR (CDCl3) 7.4-7.2 (m, 6H), 7.12 (m, 3H), 6.60 (bs, 1H), 5.05 (bs, 2H), 3.33 (bd, 2H, 15 Hz), 3.12 (d, 2H, 15 Hz), 2.85 (m, 1H), 1.20 (d, 6H, 7 Hz).

### Step 4

### [1-Ethyl-4-(3-isopropyl-phenyl)-2-oxo-piperidin-4-yl]-carbamic acid benzyl ester

0.132 g of 3-Benzyloxycarbonylamino-3-(3-isopropyl-phenyl)-5-oxo-pentanoic acid was dissolved in 10 mL DCE. 1.0 mL of 2M ethylamine in methanol was added along with 0.30 g NaBH(OAc)3. The reaction was stirred at 23 C for 2.5 hr then evaporated. The residue was stirred well in 10 mL DMF and heated to 120 C for 1 hr, allowed to cool, poured into a separatory funnel containing 50 mL EtOAc, then washed twice with 0.1M HCl, twice with 0.1 NaHCO3, brine, dried (MgSO4) and evaporated. The crude was purified by silica flash column using 9:1 EtOAc/hexane. Yield=0.061 g (45%). ESMS MH+=395.4 HNMR (CDCl3) 7.35 ppm (m, 5H), 7.29 (m, 1H), 7.15 (m, 3H), 5.60 (s, 1H), 5.00 (s, 2H), 3.48 (m, 1H), 3.25 (m, 2H), 3.05 (m, 2H), 2.85 (m, 3H), 2.25 (m, 1H), 1.20 (d, 6H, 7 Hz), 1.05 (t, 3H, 7 Hz)

### Step 4a

### [4-Benzyloxycarbonylamino-4-(3-isopropyl-phenyl)-2-oxo-piperidin-1-yl]-acetic acid methyl ester

0.0.71 g of 3-benzyloxycarbonylamino-3-(3-isopropyl-phenyl)-5-oxo-pentanoic acid was dissolved in 2.5 mL DCE. 0.070 g of glycine methyl ester hydrochloride salt was added and the flask warmed until a slightly cloudy solution was obtained. 0.10 g of NaBH(OAc)3 was added and the mixture stirred at 23 C for 3 hr. The reaction mixture was poured into 20 mL ether and 15 mL 0.1M citric acid. The layers separated and the ether layer was washed with water, brine, dried (MgSO4) and evaporated. The crude product was purified by silica flash column using 3:1 EtOAc/hexane. Yield=0.030 g (38%). ESMS MH+=439.3 HNMR (CDCl3) 7.35 ppm (m, 5H), 7.29 (m, 1H), 7.15 (m, 3H), 5.40 (s, 1H), 5.00 (s, 2H), 4.10 (dd, 2H, 7 Hz, 18 Hz), 3.70 (s, 3H), 3.40 (m, 1H), 3.2 (m, 1H), 3.05 (m, 1H), 2.85 (m 3H), 2.35 (m, 1H), 1.20 (d, 6H, 7 Hz)

### Step 5

### {1-(3,5-Difluoro-benzyl)-3-[1-ethyl-4-(3-4-isopropyl-phenyl)-2-oxo-piperidin-4-ylamino]-2-hydroxy-propyl}-carbamic acid tert-butyl ester

In a Parr hydrogenation vessel 0.061 g of [1-Ethyl-4-(3-isopropyl-phenyl)-2-oxo-piperidin-4-yl]-carbamic acid benzyl ester prepared in Step 4 was dissolved in 15 mL methanol and 30 uL acetic acid. 0.020 g of 10% Pd/C was added and the mixture rocked under 50 psi hydrogen gas for 1 hr. The mixture was filtered through a pad of celite and the filtrate evaporated on high vacuum for 2 hours. The resulting amine salt was dissolved in 0.40 mL isopropanol and transferred to a screw top vial. 0.047 g of epoxide [2-(3,5-Difluoro-phenyl)-1-oxiranyl-ethyl]-carbamic acid tert-butyl ester (4) was added along with 0.02 mL DIEA. The vial was sealed and the reaction shaken at 70° C. overnight. Another 0.020 g of epoxide (4) was added and the vial shaken at 75° C. for 2 hr. The reaction was diluted with 11.0 mL methanol and the product purified by direct injection onto RPHPLC as in Example 1 Step 7 using a gradient of 20% B->70% B. Appropriate fractions were pooled and evaporated. Yield=0.034 g ESMS MH+=560.5

### Step 6

### N-{(1S,2R)-1-(3,5-Difluoro-benzyl)-3-[1-ethyl-4-(3-isopropyl-phenyl)-2-oxo-piperidin-4-ylamino]-2-hydroxy-propyl}-acetamide

0.033 g of {1-(3,5-Difluoro-benzyl)-3-[1-ethyl-4-(3-isopropyl-phenyl)-2-oxo-piperidin-4-ylamino]-2-hydroxy-propyl}carbamic acid tert-butyl ester (The compound from step 5) was stirred in 1 mL CHCl3 and 2 mL TFA for 20 min then evaporated and placed under high vacuum for 2 hr. The residue was dissolved in 3 mL DCM and 0.10 mL DIEA. 8.0 uL acetic anhydride was added and the solution stirred at 23 C for 15 min. 10 uL of ethanolamine was added to quench excess acetic anhydride and the reaction was evaporated. The residue was purified by silica gel chromatography using a mobile phase gradient of 1% MeOH in CHCl₃→10% MeOH in CHCl₃.

In Examples 2a-2h listed below in Table 2, compounds represented by Formula V were synthesized by steps similar to those described above for Example 2 and were tested in BACE cell free assay as described in Example B. Compounds are 1:1 mixtures of diastereomers at the piperidine position.

TABLE 2
V

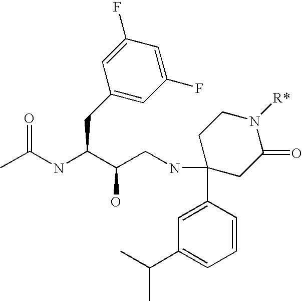

| | | MH+ observed |
|---|---|---|
| | | (expect) from |
| Example | R* | LC-ESMS |
| 2a | —CH2—CH3 | 502.36 (502.28) |
| 2b | —CH2—CO—OH | 532.21 (532.3) |
| 2c | —CH2—CO—OCH3 | 545.33 (545.3) |
| 2d | —H | 474.0 (474.3) |
| 2e | —CH2—CH2—OH | 518.2 (518.3) |
| 2f | —CH2—CH2—O-tBu | 574.3 (574.3) |
| 2g | —CH2—C6H5 | 564.3 (564.3) |
| 2h | —CH2—CO—OiPr | 574.4 (574.3) |

### Step 7

### Resolution of the Diastereomers at the Piperidine 4 Position

25 mg of N-[(1S,2R)-1-(3,5-Difluoro-benzyl)-3-[1-ethyl-4-(3 isopropyl-phenyl)-2-oxo-piperidin-4-ylamino]-2-hydroxy-propyl]-acetamide (the compound from Step 6) was dissolved in 1.0 mL methanol and purified by direct injection onto RPHPLC as in Example 1 Step 7 substituting a Vydac 20×250 mm C18 column and using a gradient of 10% B->60% B over 50 min. The diastereomers separated and appropriate fractions were pooled and evaporated. Yield of more polar, earlier eluting isomer=7.5 mg ESMS MH+=502.4 HNMR (CDCl3) 7.45 ppm (s, 1H), 7.37 (d, 1H, 8 Hz), 7.30 (m, 2H), 6.70 (d, 2H, 6 Hz), 6.64 (t, 1H), 6.46 (d, 1H, 8 Hz), 4.08 (m, 1H), 3.72 (t, 1H), 3.53 (m, 1H), 3.31 (d, 1H, 15 Hz), 3.25 (d, 1H, 6 Hz), 3.15 (m, 1H), 3.05 (m, 2H), 2.7-3.0 (5H), 2.60 (d, 2H, 11 Hz), 1.88 (s, 3H), 1.20 (dd, 6H, 6 Hz), 0.97 (t, 3H, 7 Hz). Yield of less polar, later eluting isomer=4.5 mg ESMS MH+=502.4. HNMR very similar to more polar isomer.

In Examples 2i-2n listed below in Table 3, compounds represented by Formula Va and Formula Vb were prepared by chromatographic resolution as described in Example 2 Step 7. The absolute stereochemistry of Example 2n was determined by X-ray crystallography. The absolute stereochemistry of Example 2m was assigned as opposite that of 2n. The absolute stereochemistry of Examples 2i-l has been assigned by analogy to the BACE activity of 2m and 2n as well as their relative elution times on reverse phase HPLC.

TABLE 3
V

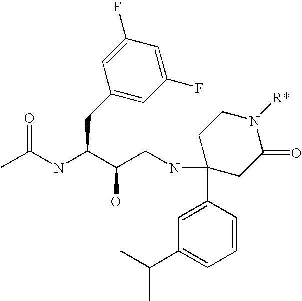

Va

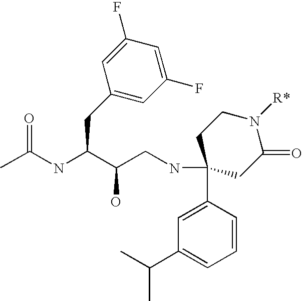

Vb

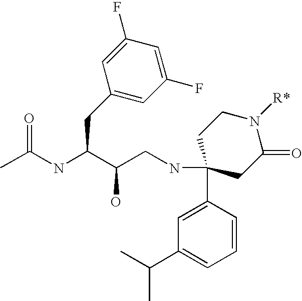

| | | Relative | Absolute |
|---|---|---|---|
| | | elution on C18 | stereochemistry at |
| Exam- | | Reverse Phase | piperidine 4-position |
| ple | R* | Chromatography | (Formula) |
| 2i | —CH₂CH₃ | First (more polar) | R (Va) |
| 2j | —CH₂CH₃ | Second (less polar) | S (Vb) |
| 2k | —CH₂—COOCH₃ | First (more polar) | R (Va) |
| 2l | —CH₂—COOCH₃ | Second (less polar) | S (Vb) |
| 2m | —CH₂—COOH | First (more polar) | R (Va) |
| 2n | —CH₂—COOH | Second (less polar) | S (Vb)* |
| 2m | —CH₂CH₂OH | First (more polar) | R (Va) |
| 2n | —CH₂CH₂OH | Second (less polar) | S (Vb) |

* determined by X-ray crystallography

### Example 3

### N-{1-(3,5-Difluoro-benzyl)-3-[1-ethyl-4-(3-isopropyl-phenyl)-2-oxo-piperidin-4-ylamino]-2-hydroxy-propyl}-acetamide

### Alternate Synthesis

Referring to Scheme 1c, intermediate compounds 14, 16, 23, 24, 25, 26, 27 and the Me ester of 17 were prepared according to the following procedures:

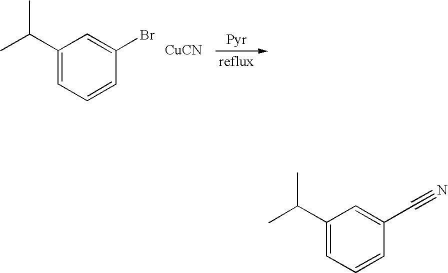

In a 250 ml round bottom flask with reflux condenser and N₂ cap, a solution of 25 gm (125.6 mmol) 1-bromo-3-isopropylbenzene and 45 gm (502.5 mmol) copper cyanide in 50 ml of pyridine was heated in a 150° C. oil bath for 2 hr where upon a dark brown homogeneous solution was formed. The reaction mixture was heated at this temperature for 11 hrs. The refluxing solution was treated with benzene and allowed to cool. The reaction mixture was partitioned between 400 ml conc NH₄OH and 200 ml of benzene and was then stirred for 30 min. The mixture was poured through a small pad of silica to remove any remaining solids. The organic phase was removed and the aqueous layer was washed several times with methylene chloride. The combined organic layers were washed with saturated brine and then dried and evaporated to afford 18 gm (100%) of 3 isopropylbenzonitrile. NMR (400 MHz, CDCl₃) δ 7.5-7.2 (m, 3H), 2.9 (m, 1H), 1.2 (d, 6H) ppm

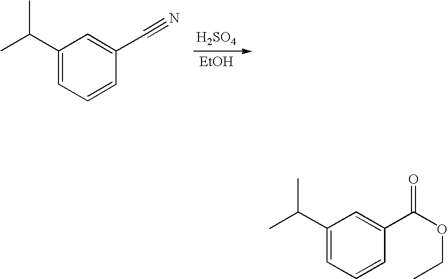

A solution of 18.3 gm (125.56 mmol) 3-isopropylbenzonitrile in 400 ml ethanol and 60 ml concentrated sulfuric acid was heated under reflux for 45 hrs. The reaction mixture was cooled to room temperature and was extracted with 3×100 ml of dichloromethane. The organic phase was evaporated in vacuo to an oil. Analysis by NMR indicated the reaction was not complete. The residue was redissolved in 600 ml of ethanol and was treated with 60 ml of concentrated sulfuric acid and heated under reflux for 20 hours. The reaction mixture was cooled to room temperature and was extracted with 3×100 ml of dichloromethane. The reaction mixture was cooled to room temperature and was extracted with 3×100 ml of dichloromethane to afford 21 gm (87%) of ethyl 3-isopropylbenzoate as an oil. NMR (400 MHz, CDCl₃) δ 7.9 (m, 2H), 7.5-7.2 (m, 2H), 4.4 (q, 2H), 2.9 (m, 1H), 1.4 (t, 3H), 1.2 (d, 6H) ppm

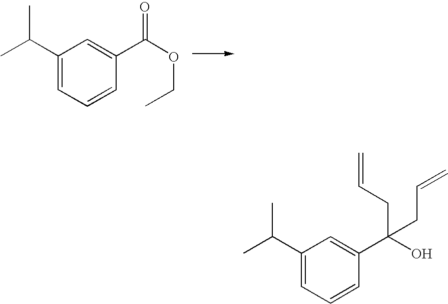

A solution of 4.79 gm (25 mmol) ethyl 3-isopropylbenzoate in THF under N₂ was treated at room temperature with 31 ml (62 mmol) of a 2M solution of allyl magnesium bromide and was stirred for 3 hrs at room temperature. The reaction mixture was heated under reflux for 1.5 hrs, allowed to cool to room temperature and then quenched with water. The mixture was extracted with dichloromethane (3×100 ml), washed with brine and then dried and evaporated. The residue was chromatographed on silica eluting with hexanelethyl acetate in a ration of 8/1. There was obtained 4.79 gm (84%) of 4-(3-isopropylphenyl)hepta-1,6-dien-4-ol. NMR (400 MHz, CDCl₃) δ 7.2 (m, 4H), 5.5 (m, 2H), 5.0 (m, 4H), 2.9 (m, 1H), 2.7 (m, 2H), 2.4 (m, 2H), 1.2 (d, 6H) ppm.

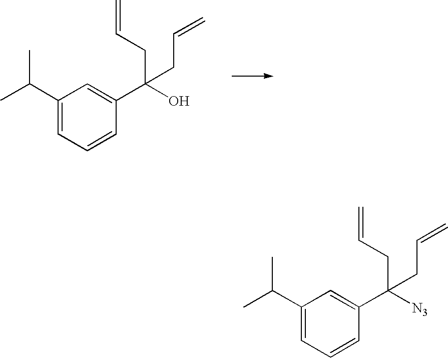

A well stirred mixture of 4.79 gm (20.8 mmol) 4-(3-isopropylphenyl)hepta-1,6-dien-4-ol and 6.5 gm (100 mmol) sodium azide in 50 ml of chloroform was cooled to −15° C. A solution of 31 ml (400 mmol) trifluoroacetic acid in 50 ml of chloroform was added drop wise over the course of an hour keeping the reaction temperature near −15° C. The mixture was stirred for an additional 2 hrs at −15° C. and then the cooling bath was removed and the reaction mixture was allowed to warm to room temperature overnight. The reaction was quenched by the addition of water and the product was isolated after extraction of the aqueous layer three times with dichloromethane. The organic layer was washed with brine and then dried and evaporated. After evaporation of the solvent the residue was chromatographed on silica eluting with hexane/ethyl acetate (10/1) to afford 3.48 gm (65%) of 1-(4-azidohepta-1,6-dien-4-yl)-isopropylbenzene. NMR (400 MHz, CDCl₃) δ 7.2 (m, 4H), 5.6 (m, 2H), 5.0 (m, 4H), 2.9 (m, 1H), 2.7 (m, 4H), 1.3 (d, 6H) ppm.

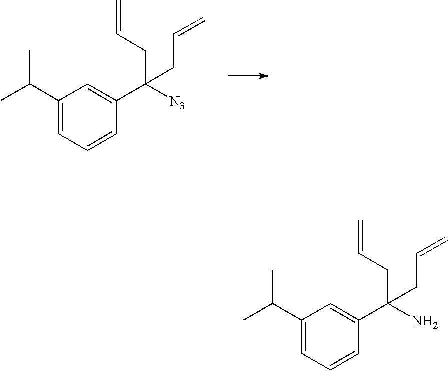

To a solution of the 2.0 gm (7.8 mmol) azide, prepared above, in 20 ml of THF was added water (0.28 ml (15.5 mmol) and 9.4 ml (9.4 mmol) of a 1 M solution of trimethylphosphine in THF. The reaction mixture was stirred for 20 hrs before water (20 ml) was added and the reaction mixture was stirred for an additional 2 hrs. The mixture was extracted three times with 50 ml of methylene chloride. The organic layer was washed with brine and then dried and evaporated. The residue (approximately 1.8 gms) was used directly in the next step.

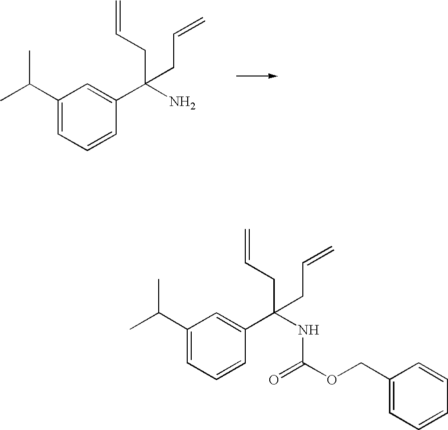

The material from the previous step (approximately 7.8 mmol) was dissolved in 15 ml of dioxane-water (60/40) and the solution was cooled to 0° C. Sodium bicarbonate (5.9 gm, 70.6 mmol) and 6.7 ml (47 mmol) of cbz-cl were then added to the well-stirred mixture in sequence. The reaction mixture was stirred for 24 hrs at room temperature and then at 50° C. for 2 hrs. The mixture was cooled and then extracted with 3×50 ml of methylene chloride. The organic layer was washed with brine and then dried and evaporated. The residue was chromatographed on silica eluting with hexane/ethyl acetate (6/1) to afford 2.05 gm (72%) of benzyl 4-(3-isopropylphenyl)hepta-1,6-dien-4-ylcarbamate. NMR (400 MHz, CDCl₃) δ 7.4 (m, 2H), 7.1 (m, 2H), 5.6 (m, 2H), 5.0 (m, 6H), 2.9 (m, 3H), 2.7 (m, 2H), 1.3 (d, 6H) ppm

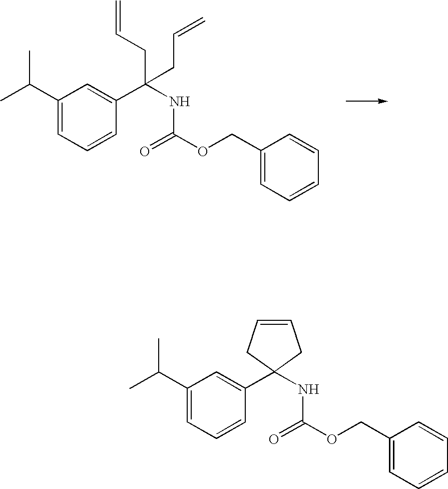

To a three neck round bottom flask was added 3.15 gm (8.66 mmol) of benzyl 4-(3-isopropylphenyl)hepta-1,6-dien-4-ylcarbamate from above, 0.5 gm (0.61 mmol) of the Grubbs catalyst (1^{st} generation) and 70 ml of benzene. The mixture was heated to 40° C. and degassed with nitrogen and vacuum cycles. The mixture was stirred at 40° C. for 14 hrs. The reaction was monitored by t/c and it was determined that a significant amount of starting material remained. The reaction was once again degassed with several nitrogen and vacuum cycles and allowed to stir for 2 additional hours at 40° C. The reaction mixture was quenched with water and then extracted with 3×50 ml of methylene chloride. The organic layer was washed with brine and then dried and evaporated. The residue was chromatographed on-silica eluting with hexane/ethyl acetate (8/1) to afford 1.79 gm (59%) of the desired product benzyl 1-(3-isopropylphenyl)cyclopent-3-enylcarbamate. Also obtained was 1.09 gm (35%) of the starting material. NMR (400 MHz, CDCl₃) δ 7.4 (br.s, 1H), 7.2-7.1 (m, 4H), 5.8 (s, 1H), 5.3 (s, 1H), 5.0 (s, 2H), 2.9 (m, 5H), 1.3 (d, 6H) ppm.

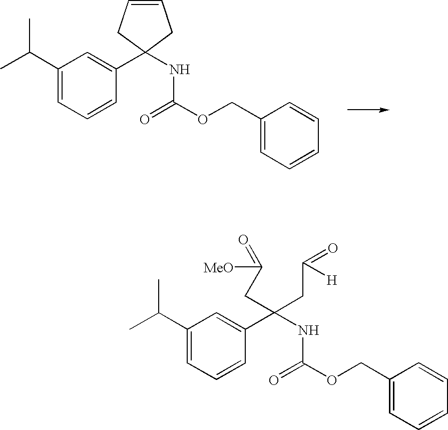

To 1.7 gm (5.07 mmol) of cyclopentene in 100 ml of dichloromethane-methanol (80/20) was mixed with 1.7 gm (20 mmol) sodium bicarbonate followed by cooling to −78° C. The reaction mixture was treated with a stream of ozone in oxygen until the solution turned a dark blue. The ozone was discharged with nitrogen and the reaction mixture was warmed and quenched with water followed by extraction with 3×50 ml of methylene chloride. The organic layer was washed with brine and then dried and evaporated. The residue was redissolved in dichloromethane and was treated with 1.06 ml (7.6 mmol) triethylamine and 1.44 ml (15.2 mmol) acetic anhydride and stirred for 18 hrs. The reaction mixture was quenched with water and then extracted with 3×50 ml of methylene chloride. The organic layer was washed with brine and then dried and evaporated. The residue was chromatographed on silica eluting with hexane/ethyl acetate (4/1) to afford 0.62 gm (31%) of an oil. NMR (400 MHz, CDCl₃) δm 9.7 (s, 1H), 7.4-7.1 (m, 5H), 5.1 (m, 2H), 3.5 (s, 3H), 3.1 (m, 4H), 2.9 (m, 1H), 1.3 (d, 6H) ppm. This product can be substituted for 3-benzyloxycarbonylamino-3-(3-isopropyl-phenyl)-5-oxo-pentanoic acid in step 4 of example 2. Further processing according to example 2 affords N-{1-(3,5-Difluoro-benzyl)-3-[1-ethyl-4-(3-isopropyl-phenyl)-2-oxo-piperidin-4-ylamino]-2-hydroxy-propyl}-acetamido.

### Example 4

The following compounds were prepared using 4-(3-tert-butyl-phenyl)-1-pyrimidin-2-yl-piperidin-4-ylamine and 4-(3-tert-butyl-phenyl)-1-thiazol-2-yl-piperidin-4-ylamine from Preparations 5 and 6 using the methods described in Steps 4 and 5 of Example 1:

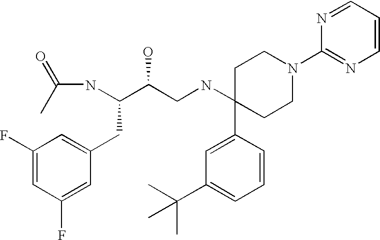

(4a) N-[(1S,2R)-3-[4-(3-tert-Butyl-phenyl)-1-pyrimidin-2-yl-piperidin ylamino]-1-(3,5-difluoro-benzyl)-2-hydroxy-propyl]-acetamide: APCl⁺ MS-MH⁺ observed 552.5, expected 552.3;

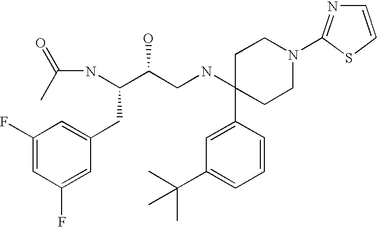

(4b) N-[(1S,2R)-3-[4-(3-tert-Butyl-phenyl)-1-thiazol-2-yl-piperidin-4-ylamino]-1-(3,5-difluoro-benzyl)-2-hydroxy-propyl]-acetamide: APCl⁺MS-MH⁺ observed 557.5, expected 557.3

### Example 5

### 3-[(2R,3S)-3-Acetylamino-4-(3,5-difluoro-phenyl)-2-hydroxy-butylamino]-3-(3-isopropyl-phenyl)-azetidine-1-carboxylic acid benzyl ester

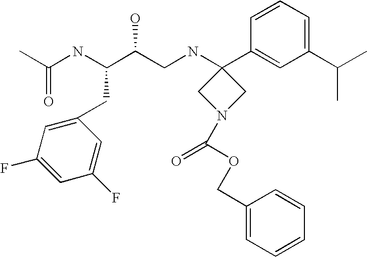

A solution of 3-[(2R,3S)-3-amino-4-(3,5-difluoro-phenyl)-2-hydroxy-butylamino]-3-(3-isopropyl-phenyl)-azetidine-1-carboxylic add benzyl ester hydrochloride salt (Preparation 14, 0.362 g, 0.691 mmol) and N-methylmorpholine (0.299 mL, 3.11) in CH₂Cl₂ (3.5 mL) was stirred at 0° C. for 15 min. Acetic add (0.044 mL, 0.760 mmol) was added and after stirring 5 min, 1-hydroxybenzotriazole (0.103 g, 0.760 mmol) and 1-(3-dimethylaminopropyl)-3-ethylcarbodiimide hydrochloride (0.146 g, 0.760 mmol) were added and the resulting mixture was stirred at rt for 16 h. The reaction was diluted with CH₂Cl₂ and washed with sat. NaHCO₃, water and brine, dried (MgSO₄) and concentrated to yield 355 mg of a sticky oil. Chromatography, eluting with 200 mL hexanes, 500 mL EtOAc and 500 mL 10% MeOH/EtOAc, yielded 262 mg (67%) of the title compound: NMR (CDCl₃) δ 7.34-7.25 (m, 6H), 7.16-7.14 (d, J=7.9 Hz, 1H), 7.09-7.05 (m, 2H), 6.65-6.60 (m, 3H), 5.93 (d, J=8.7 Hz, 1H), 5.09 (s, 2H), 4.36-4.30 (m, 2H), 4.13-4.02 (m, 3H), 3.41-3.39 (m, 1H), 2.93-2.72 (m, 2H), 2.70-2.66 (m, 1H), 2.46-2.38 (br m, 2H), 1.82 (s, 3H), 1.23 (d, J=7.1 Hz, 6H); LCMS MH⁺=566.1

### Example 6

### N-[(1S,2R)-3-[1-Acetyl-3-(3-isopropyl-phenyl)-azetidin-3-ylamino]-1-(3,5-difluoro-benzyl)-2-hydroxy-propyl]-acetamide

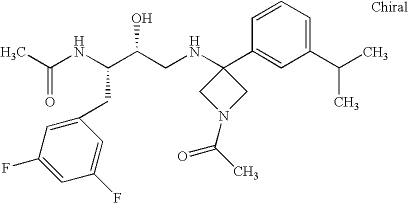

Acetyl chloride (0.005 mL, 0.070 mmol) was added to a solution of N-{(1S,2R)-1-(3,5-difluoro-benzyl)-2-hydroxy-3-[3-(3-isopropyl-phenyl)-azetidin-3-ylamino]-propyl}acetamide (Preparation 15, 0.030 g, 0.070 mmol), and diisopropylethyl amine (0.012 mL, 0.070 mmol) in CH₂Cl₂ (1 mL). After stirring for 3 days, the mixture was washed with water and brine, dried (Na₂SO₄) and concentrated to a yield 29 mg of crude product. Chromatography using a 50% EtOAc/hexanes to 20% MeOH/EtOAc gradient yielded 2.8 mg of title compound: LCMS MH⁺=474.2

The following compounds, prepared by the general procedure described in Example 6, had BACE IC₅₀ values of less 3,000 nM.

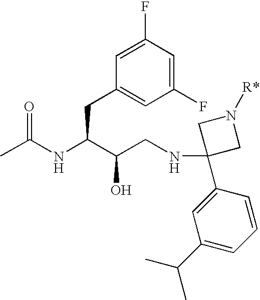

| | | Observed LCMS MH+ |
|---|---|---|
| Example | R* | (Expected) |
| 6a | —CO—CH₂—CO—OCH₃ | 532.2 |
| | | (532.3) |
| 6b | —CO—OCH₃ | 490.1 |
| | | (490.3) |

### Example 7

The following compounds were prepared using N-{(1S,2R)-1-(3,5-difluoro-benzyl)-2-hydroxy-3-[3-(3-isopropyl-phenyl)-pyrrolidin-3-ylamino]-propyl}-acetamide (Preparation 21) by essentially the same procedure described in Example 6. The diastereomers were then separated by chromatography on a Kromasil DMB column (5 cm×25 cm) using 90% heptane/isopropyl alcohol or 92% heptane/EtOH with 0.1% TFA: Compounds 7a-7g had BACE IC₅₀ values of less 10,000 nM.

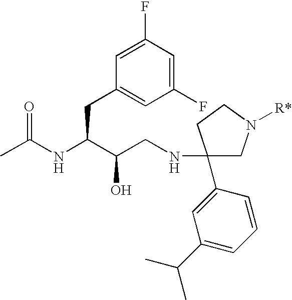

| | | Observed LCMS MH+ |
|---|---|---|
| Example | R* | (Expected) |
| diastereomer 1 | —CO—CH₃ | 488.1 (488.3) |
| 7a | | |
| diastereomer 1 | —CO—OCH₃ | 504.1 (504.3) |
| 7b | | |
| diastereomer 2 | —CO—OCH₃ | 504.1 (504.3) |
| 7c | | |
| diastereomer 1 | —SO₂CH₃ | 524.1 (524.3) |
| 7d | | |
| diastereomer 2 | —SO₂CH₃ | 524.1 (524.3) |
| 7e | | |
| diastereomer 1 | —CO—CH₂—CO—OCH₃ | 546.5 (546.3) |
| 7f | | |
| diastereomer 2 | —CO—CH₂—CO—OCH₃ | 546.5 (546.3) |
| 7g | | |

### Example 8

The following compounds, 8a-8f, were prepared as mixtures of diastereomers using compounds from preparations 27-29 following the method described in Example 6. They had BACE IC₅₀ values of less than 10,000 nM.

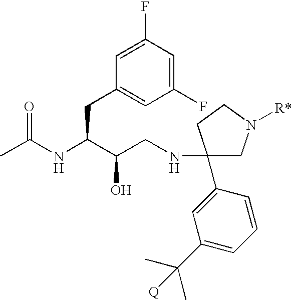

| | | Observed LCMS MH+ |
|---|---|---|
| Example | Compound | (Expected) |
| 8a | R* = pyridin-2-yl | 523.5 (523.3) |
| | Q = H | |
| 8b | R* = benzooxazol-2-yl | 563.5 (563.3) |
| | Q = H | |
| 8c | R* = thiazol-2-yl | 529.3 (529.2) |
| | Q = H | |
| 8d | R* = pyrimidin-2-yl | 524.4 (524.3) |
| | Q = H | |
| 8e | R* = 5-bromo-pyrimidin-2-yl | 616.2, 618.2 |
| | Q = CH₃ | (616.2, 618.2) |
| 8f | R* = 4-methoxy-pyrimidin-2-yl | 568.2 (568.3) |
| | Q = CH₃ | |

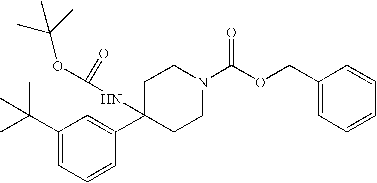

### Preparation 1

### 4-tart-Butoxycarbonylamino-4-(3-tort-butyl-phenyl)-piperidin-1-carboxylic acid benzyl ester

4-Amino-4-(3-tert-butyl-phenyl)-piperidine-1-carboxylic acid benzyl ester (prepared as in Step 3 of Example 1) (0.50 g, 1.36 mmol) in THF (10 mL) was added to a slurry of sodium hydride (0.15 g, 3.75 mmol) in THF (10 mL). After stirring 5 min, di-t-butyl dicarbonate (0.41 g, 1.88 mmol) in THF (5 mL) was added and the mixture was refluxed for 22 hrs. The reaction was cooled, quenched with water and concentrated. The residue was partitioned between EtOAc and water, the organics were washed with brine, dried (MgSO₄) and concentrated to yield a straw colored oil. Chromatography using 10-20% EtOAc/hexanes yielded 0.466 g (74%) of the title compound as a white foam: NMR (CDCl₃) δ 7.49-7.27 (m, 5H), 7.25-7.21 (m, 3H), 7.15-7.11 (m, 1H), 5.13 (s, 2H), 4.78 (s, 1H), 4.17-3.93 (m, 2H), 3.28-3.08 (m, 2H), 2.40-2.08 (br m, 2H), 2.00-1.88 (m, 2H), 1.60-1.00 (m, 18H).

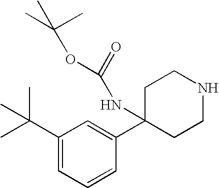

### Preparation 2

### [4-(3-tert-Butyl-phenyl)-piperidin-4-yl]-carbamic acid tert-butyl ester

4-tert-Butoxycarbonylamino-4-(3-tert-butyl-phenyl)-piperidine-1-carboxylic acid benzyl ester (Preparation 1) (0.466 g, 1.00 mmol), and 10% palladium on carbon (75 mg) in EtOH (30 mL) where shaken at rt under 47 psi hydrogen for 6 h. The mixture was filtered (Celite)ntrated to afford 0.344 g of title compound as a sticky yellow foam: NMR (CDCl₃) δ 7.39 (s, 1H), 7.25-7.23 (m, 2H), 7.18-7.13 (m, 1H), 4.77 (br s, 1H), 3.42-3.18 (m, 4H), 2.46 (br s, 4H), 1.50-1.00 (m, 18H).

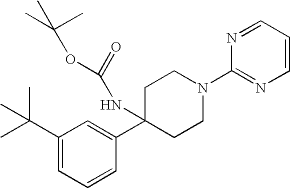

### Preparation 3

### [4-(3-tert-Butyl-phenyl)-1-pyrimidin-2-yl-piperidin-4-yl]-carbamic acid tert-butyl ester

[4-(3-tert-Butyl-phenyl)-piperidin-4-yl]-carbamic acid tert-butyl ester (Preparation 2) (0.200 g, 0.602 mmol), 2-chloropyrimidine (0.21 g, 1.83 mmol), K₂HPO₄ (0.31 g, 1.78 mmol) and DMSO (2 mL) were heated in a sealed tube at 90° C. for 24 hrs, cooled, diluted with half-saturated NaCl solution and extracted into ˜100 mL ether. The extract was dried (MgSO₄) and concentrated. Excess 2-chloropyrimidine was removed by sublimation with heating under vacuum to leave 0.21 g (86%) of the title compound as a yellow-brown oil: NMR (CDCl₃) δ 8.63 (d, J=4.6 Hz, 1H), 8.30-8.29 (m, 2H), 7.41 (s, 1H), 7.34-7.14 (m, 2H), 6.47 (t, J=4.8 Hz, 1H), 4.90 (s, 1H), 4.62 (br d, J=13.7 Hz, 2H), 3.29-3.20 (m, 2H), 2.50-2.10 (m, 2H), 2.03 (dt, J=13.1, 4.0 Hz, 2H), 1.60-1.0 (m, 18H).

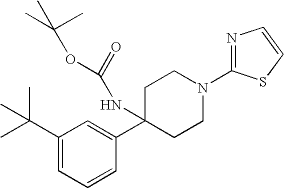

### Preparation 4

### [4-(3-tort-Butyl-phenyl)-1-thiazol-2-yl-piperidin-4-yl]-carbamic acid tert-butyl ester

This compound was prepared by the method described in Preparation 3, substituting 2-bromothiazole for 2-chloropyrimldlne: yield 83%; NMR (CDCl₃) δ 7.58 (d, J=3.8 Hz, 1H), 7.41 (s, 1H), 7.28-7.14 (m, 3H), 6.56 (d, J=3.7 Hz, 1H), 4.84 (s, 1H), 2.89 (br d, J=12.0 Hz, 2H), 3.36 (br t, J=12.2 Hz, 2H), 2.45-2.20 (m, 2H), 2.17 (dt, J=13.1, 4.6 Hz, 2H), 1.45-1.00 (m, 18H).

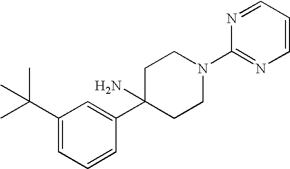

### Preparation 5

### 4-(3-tert-Butyl-phenyl)-1-pyrimidin-2-yl-piperidin-4-ylamine

4N HCl in dioxane (4 mL) was added to a solution of [4(3-trt-butyl-phenyl)-1-pyrimidin-2-yl-piperidin-4-yl]-carbamic acid tert-butyl ester (from Preparation 3, 0.21 g, 0.51 mmol) in dioxane (4 mL) and the resulting mixture was stirred for 19 hrs at rt. Ether (20 mL) was added to precipitate an orange tinged solid which was collected then partitioned between EtOAc and aq. K₂CO₃. The organics were washed with brine, dried (MgSO₄) and concentrated to yield 0.119 g (75%) of the title compound as an orange oil: NMR (CDCl₃) δ 8.28 (d, J=5.0 Hz, 2H), 7.49 (t, J=1.5 Hz, 1H), 7.29-7.25 (m, 3H), 6.43 (t, J=4.8 Hz, 1H), 4.40 (dt, J=13.7, 3.7 Hz, 2H), 3.61-3.53 (sym. mult, 2H), 2.15-2.08 (sym. mult., 2H), 1.77 (br d, J=12.9 Hz, 2H), 1.55 (br s, 2H), 1.30 (s, 9H).

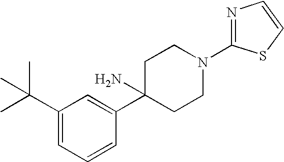

### Preparation 6

### 4-(3-tert-Butyl-phenyl)-thiazol-2-yl-piperidin-4-ylamine

This compound was prepared by the method described in Preparation 5 using the compound from Preparation 4: yield 59%; NMR (CDCl₃) δ 7.48 (s, 1H), 7.31-7.20 (m, 3H), 7.17 (d, J=3.7 Hz, 1H), 6.52 (d, J=3.7 Hz, 1H), 3.79-3.73 (m, 2H), 3.62-3.55 (sym.mult., 2H), 2.26-2.19 (sym.mult., 2H), 1.80 (br d, J=12.9 Hz, 2H), 1.45 (br s, 2H), 1.31 (s, 9H).

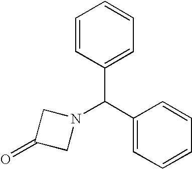

### Preparation 7

### 1-Benzhydryl-azetidin-3-one

Trifluoroacetic anhydride (7.07 mL, 50.1 mmol) was added to a −78° C. solution of DMSO (4.75 mL, 66.9 mmol) in CH₂Cl₂ (150 mL). After 15 min, 1-benzhydryl-azetidin-3-ol (27) (8.0 g, 33.4 mmol) in CH₂Cl₂ (150 mL) was added dropwise over 20 min. The mixture was stirred for 1 hr, then diisopropylethylamine (25.7 mL, 147.0 mmol) was added, the mixture was stirred 15 min and subsequently warmed to rt. Saturated aq. NH₄Cl was added and the mixture was extracted into EtOAc and washed with brine. Concentration yielded an orange oil which was purified by chromatography using 10% EtOAc/hexanes as eluent to yield 5.11 g (64%) of the title compound as a yellow solid: mp 70-73° C.; NMR (CDCl₃) δ 7.52-7.44 (m, 4H), 7.33-7.20 (m, 6H), 4.61 (s, 1H), 4.00 (s, 4H); ¹³C NMR (CDC₃) δ 201.28, 142.66, 128.99, 127.78, 127.52, 78.00, 74.47.

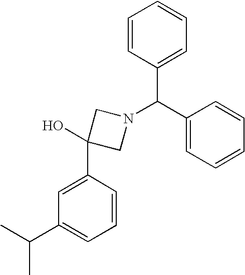

### Preparation 8

### 1-Benzhydryl-3-(3-isopropyl-phenyl)-azetidin-3-ol

Magnesium turnings (2.50 g, 103 mmol) and a few flakes of iodine were heated until the iodine sublimed. After cooling to rt, a solution of 3-bromoisopropylbenzene (9.35 g, 46.8 mmol) and 1,2-dibromoethane (3 drops) in ether (78 mL) was added. The mixture was refluxed for 1 hr and cooled to rt. 1-Benzhydryl-azetidin-3-one (from Preparation 7, 5.55 g, 23.3 mmol) in ether (230 mL) was added dropwise over 15 min. After addition, the reaction was stirred for 10 min during which time a precipitate formed. Saturated aq. NH₄Cl was added, the organics were separated and the aq. phase was re-extracted with ether. The combined organics were washed with brine, dried (Na₂SO₄) and concentrated to yield an orange oil. Chromatography with 15% EtOAc/hexanes afforded 7.22 g (87%) of the title compound as a yellow oil: NMR (CDCl₃) δ 7.49-7.19 (m, 14H), 4.51 (s, 1H), 3.62 (d, J=8.3 Hz, 2H), 3.45 (d, J=8.3 Hz, 2H), 3.15 (br s, 1H), 2.96 (hept, J=6.9 Hz, 1H), 1.30 (d, J=7.1 Hz, 6H); ¹³C NMR (CDCl₃) δ 149.36, 144.36, 142.40, 128.76, 127.74, 127.44, 125.86, 123.43, 122.79, 78.25, 71.91, 67.68, 66.13, 34.53, 24.34, 15.54.

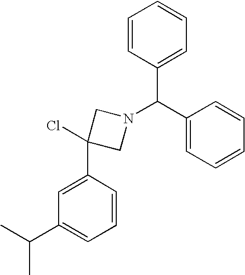

### Preparation 9

### 1-Benzhydryl-3-chloro-3-(3-isopropyl-phenyl)-azetidine

1-Benzhydryl-3-(3-isopropyl-phenyl)-azetidin-3-ol (Preparation 8, 6.95 g, 19.44 mmol), triethylamine (11.1 mL, 79.7 mmol) and methanesulfonyl chloride (6.17 mL, 79.7 mmol) in CHCl₃ (160 mL) were refluxed for 4 hrs and cooled to rt. Water was added and the mixture was extracted twice with CH₂Cl₂. The extracts were washed with brine, dried (Na₂SO₄) and concentrated to afford an orange oil. Chromatography using 100% hexanes and 5-10% EtOAc/hexanes yielded 3.439, (47%) of title compound as a yellow oil: NMR (CDCl₃) δ 7.45-0.715 (m, 14H), 4.46 (s, 1H), 3.88 (d, J=10 Hz, 2H), 3.76 (d, J=10 Hz, 2H), 2.88 (hept, J=6.9 Hz, 1H), 1.22 (d, J=7.1 Hz, 6H); ¹³C NMR (CDCl₃) δ 150.36, 144.38, 142.70, 129.44, 128.28, 128.19, 126.95, 124.68, 123.82, 78.26, 663.56, 34.55, 24.30.

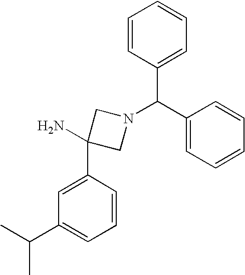

### Preparation 10

### 1-Benzhydryl-3-(3-isopropyl-phenyl)-azetidin-3-ylamine

Step 1:

1-Benzhydryl-3-chloro-3-(3-isopropyl-phenyl)-azetidine (from Preparation 9, 3.07 g, 8.41 mmol) and sodium azide (2.18 g, 33.6 mmol) in DMF (50 mL) were heated for 16 hrs at 100° C. The mixture was cooled and partitioned between water and CH₂Cl₂. The organics were: washed 3× with water and then brine, dried (Na₂SO₄) and concentrated. To remove residual DMF, the material was redissolved in EtOAc and washed 3× with water then brine and concentrated as before to yield 2.96 g of crude 3-azido-1-benzhydryl-3-(3-isopropyl-phenyl)-azetidine (31) as a yellow oil.

Step 2:

The crude azide from Step 1 (1.72 g, 10.8 mmol) was hydrogenated with 10% palladium on carbon (0.3 g) in EtOH (23 mL) under 25 PSI hydrogen for 16 h, filtered (Celite) and concentrated to afford a yellow oil. Chromatography with hexanes and 50% EtOAc/hexanes yielded 794 mg (49%) of title compound as a yellow oil: NMR (CDCl₃) δ 7.51-7.50 (m, 4H), 7.40-7.17 (m, 10H), 4.46 (s, 1H), 3.55 (d, J=8.3 Hz, 2H), 3.35 (d, J=7.9 Hz, 2H), 2.95 (hept, J=6.8 Hz, 1H), 2.12 (br s, 2H), 1.30 (d, J=6.6 Hz, 6H); ¹³C NMR (CDCl₃) δ 149.35, 146.43, 142.67, 128.73, 127.76, 127.39, 125.18, 123.70, 123.01, 78.25, 68.12, 54.56, 34.54, 24.36.

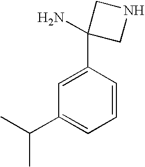

### Preparation 11

### 3-(3-Isopropyl-phenyl)-azetidin-3-ylamine

1-Benzhydryl-3-(3-isopropyl-phenyl)-azetidin-3-ylamine (from Preparation 10, 0.61 g, 1.71 mmol) in MeOH was saturated with HCl gas and concentrated. This material was re-dissolved in fresh MeOH (20 mL) and combined with 20% Pd(OH)₂ on carbon (250 mg) and hydrogenated under 40 psi hydrogen for 14 hr at rt. The mixture was filtered (Celite) and concentrated. The resulting residue was triturated with ether and filtered to yield 406 mg (90%) of the hydrochloride salt of the title compound as an orange solid: NMR (DMSO-d₆) δ 7.49 (s, 1H), 7.39-7.35 (m, 2H), 7.31-7.29 (m, 1H), 4.58 (d, J=12 Hz, 2H), 4.43 (d, J=12 Hz, 2H), 2.89 (hept, J=6.8 Hz, 1H), 1.19 (d, J=7.1 Hz, 6H).

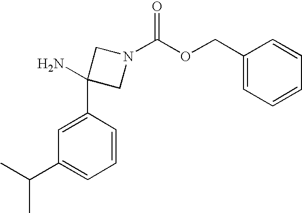

### Preparation 12

### 3-Amino-3-(3-isopropyl-phenyl)-azetidine-1-carboxylic acid benzyl ester

3-(3-Isopropyl-phenyl)-azetidin-3-ylamine hydrochloride salt (Preparation 11, 0.866 mg, 3.53 mmol) and triethylamine (1.39 mL, 9.98 mmol) were stiffed for 10 min in THF (10 mL). Benzylchloroformate (0.47 mL, 3.33 mmol) in THF (6 mL) was added and the mixture was stirred for 16 h. After concentration, the mixture was re-dissolved in EtOAc and washed with sat. aq. NaHCO₃, water and brine, dried (MgSO₄) and concentrated. Chromatography with first hexanes and then EtOAc yielded 0.443 g, (41%) of the title compound as an orange oil: NMR (CDCl₃) δ 7.34-7.13 (m, 9H), 5.10 (s, 2H), 4.39 (d, J=9.1 Hz, 2H), 4.05 (d, J=8.7 Hz, 2H), 2.89 (hept, J=6.9 Hz, 1H), 1.23 (d, J=6.6 Hz, 6H).

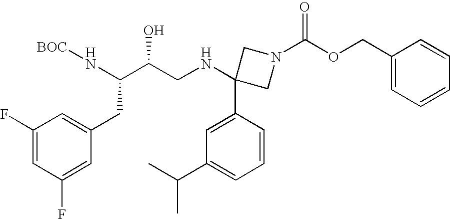

### Preparation 13

### 3-[(2R,3S)-3-tert-Butoxycarbonylamino-4-(3,5-difluoro-phenyl)-2-hydroxy-butylamino]-3-(3-isopropyl-phenyl)-cyclobutanecarboxylic acid benzyl ester

3-Amino-3-(3-isopropyl-phenyl)-azetidine-1-carboxylic acid benzyl ester (Preparation 12, 0.443 g, 1.37 mmol) and epoxide of formula 4 (0.490 g, 1.64 mmol) in isopropanol (2 mL) were refluxed for 6 hrs. An additional ˜0.5 g portion of epoxide was added and the reflux was continued for an additional 16 h. The reaction mixture was concentrated and chromatographed, eluting with hexanes and then 40% EtOAc/hexanes to afford 0.429 g (50%) of the title compound as a white solid: NMR (CDCl₃) δ 7.35-7.25 (m, 6H), 7.13 (d, J=7.9 Hz, 1H), 7.08-7.05 (m, 2H), 6.69-6.61 (m, 3H), 5.10 (s, 2H), 4.45 (d, J=9.1 Hz, 1H), 4.36-4.31 (m, 2H), 4.14-4.08 (m, 3H), 3.68 (br s, 1H), 3.28 (br s, 2H), 2.93-2.85 (m, 2H), 2.73-2.67 (m, 1H), 2.47 (br s, 2H), 1.32 (s, 9H), 1.22 (d, J=7.0 Hz, 6H); LCMS MH⁺ 624.2

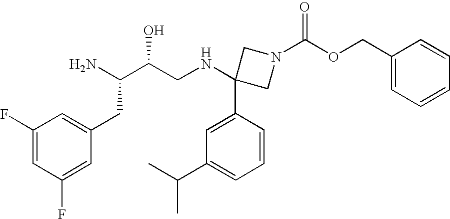

### Preparation 14

### 3-[(2R,3S)-3-Amino-4-(3,5-difluoro-phenyl)-2-hydroxy-butylamino]-3-(3-isopropyl-phenyl)-azetidine-1-carboxylic acid benzyl ester

3-[(2R,3S)-3-tert-Butoxycarbonylamino-4-(3,5-difluoro-phenyl)-2-hydroxy-butylamino]-3-(3-isopropyl-phenyl)-cyclobutanecarboxylic acid benzyl ester (Preparation 13, 0.429 g, 0.687 mmol) and 4M HCl/dioxane (8 mL) were stirred for 5 h at rt. The crude mixture was concentrated, redissolved in EtOAc and washed with sat. NaHCO₃ and brine, dried (MgSO₄) and re-concentrated to yield 362 mg (100%) of the title compound as a sticky oil: NMR (CDCl₃) δ 7.33-7.27 (m, 6H), 7.15-7.07 (m, 3H), 6.65-6.61 (m, 3H), 5.08 (m, 3H), 4.35-4.32 (br m, 2H), 4.09 (d, J=8.7 Hz, 2H), 3.37 (br s, 1H), 3.02-2.99 (m, 1H), 2.88 (hept, J=6.9 Hz, 1H), 2.71 (dd, J=13.7, 3.7 Hz, 1H), 2.58 (dd, J=11.6, 3.1 Hz, 1H), 2.47-2.44 (m, 1H), 2.37 (dd, J=13.7, 9.7 Hz, 1H), 1.21 (d, J=6.6 Hz, 6H); LCMS MH⁺ 524.2

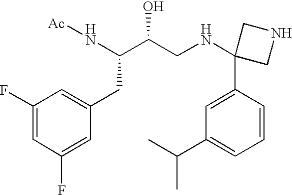

### Preparation 15

### N-{(1S,2R)-1-(3,5-Difluoro-benzyl)-2-hydroxy-3-[3-(3-isopropyl-phenyl)-azetidin-3-ylamino]-propyl}-acetamide

3-[(2R,3S)-3-Acetylamino-4-(3,5-difluoro-phenyl)-2-hydroxy-butylamino]-3(3-isopropyl-phenyl)-azetidine-1-carboxylic acid benzyl ester (Preparation 14, 0.262 g, 0.463 mmol), 10% Pd on carbon (100 mg) and ethanol (5 mL) were shaken under 40 psi of hydrogen for 5.5 h at rt. The mixture was filtered (Celite) and concentrated to yield 175 mg of the title compound as a gray solid: LCMS MH⁺ 432.1

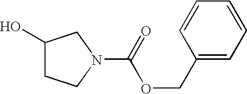

### Preparation 16

### 3-Hydroxy-pyrrolidine-1-carboxylic acid benzyl ester

3-Pyrrolidinol (6.99 g, 68.9 mmol), benzyl chloroformate (10.66 mL, 75.8 mmol) and CH₂Cl₂ (200 mL) were stirred for 20 h then washed with water and brine, dried (MgSO₄) and concentrated to give a thick yellow oil. Chromatography, flushing first with 20% EtOAc/hexanes then eluting with 30% MeOH/EtOAc, afforded 7.40 g (49%) of the title compound: NMR (CDCl₃) δ 7.35-7.26 (m, 5H), 5.11 (s, 2H), 4.46 (br s, 1H), 3.60-3.38 (m, 4H), 2.00-2.82 (m, 2H).

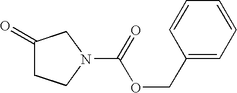

### Preparation 17

### 3-Oxo-pyrrolidine-1-carboxylic acid benzyl ester

This compound was prepared from 3-hydroxy-pyrrolidine-1-carboxylic acid benzyl ester (Preparation 16) following the same general procedure used in Preparation 7: yield 74%; NMR (CDCl₃) δ 7.34-7.28 (m, 5H), 5.14 (s, 2H), 3.83-3.78 (m, 4H), 2.56 (t, J=7.9 Hz, 2H).

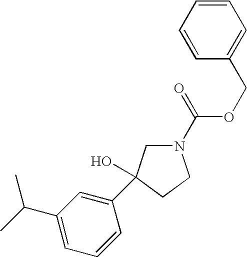

### Preparation 18

### 3-Hydroxy-3-(3-isopropyl-phenyl-pyrrolidine-1-carboxylic acid benzyl ester

Magnesium turnings (1.63 g, 67.1 mmol) and a few flakes of iodine were heated until the iodine sublimed. After cooling to rt, a solution of 3-bromoisopropylbenzene (6.09 g, 30.5 mmol) and 2 drops of 1,2-dibromoethane in ether (50 mL) was added. The mixture was refluxed for 1 hr and cooled to rt. Oxo-pyrrolidine-1-carboxylic acid benzyl ester (Preparation 7, 3.34 g, 15.3 mmol) in ether (150 mL) was added dropwise over 15 min. After addition, the reaction was stirred for 10 min during which time a white precipitate formed. Saturated aq. NH₄Cl was added, the organics were separated and the aq. phase was re-extracted with ether. The combined organics were washed with brine, dried (Na₂SO₄) and concentrated to yield a yellow oil. Chromatography with 30% EtOAc/hexanes afforded 4.14 g (80%) of the title compound as a yellow oil: NMR (CDCl₃) δ 7.37-7.15 (m, 9H), 5.16-5.04 (m, 2H), 3.83-3.63 (m, 4H), 2.90 (hept, J=6.8 Hz, 1H), 2.31-2.13 (m, 2H), 1.23 (d, J=7.1 Hz, 6H).

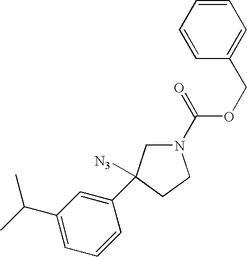

### Preparation 19

### 3-Azido-3-(3-isopropyl-phenyl)-pyrrolidine-1-carboxylic acid benzyl ester

3-Hydroxy-3-(3-isopropyl-phenyl-pyrrolidine-1-carboxylic acid benzyl ester (Preparation 18, 0.909 g, 2.68 mmol) was dissolved in a mixture of trifluoroacetic acid (9.7 mL) and water (1.6 mL) and cooled to 0° C. Sodium azide (1.20 g, 18.5 mmol) was added and the mixture was stirred for 3 h at rt., then excess NH₄OH was added and the mixture was extracted with CH₂Cl₂. The extract was washed with brine, dried (MgSO₄) and concentrated to yield 0.912 g of title compound with ˜25% of elimination by-product. This material was used without purification: NMR (CDCl₃) δ (azide product) 7.43-7.10 (m, 9H), 5.20-5.15 (m, 2H), 3.77-3.57 (m, 4H), 2.97-2.80 (m, 1H), 2.48-2.26 (m, 2H), 1.25-1.22 (m, 6H).

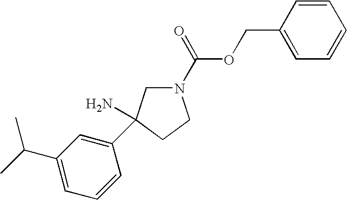

### Preparation 20

### 3-Amino-(3-isopropyl-phenyl)-pyrrolidine-1-carboxylic acid benzyl ester

Step 1:

3-Azido-3-(3-isopropyl-phenyl)-pyrrolidine-1-carboxylic acid benzyl ester (Preparation 19, 5.40 g, 14.8 mmol) was shaken under 20 psi hydrogen in the presence of 10% palladium on carbon (500 mg) in EtOH (400 mL) for 16 h. After filtration (Celite), the reaction was concentrated to yield 3.62 g of 3-(3-isopropyl-phenyl)-pyrrolidin-3-ylamine as a yellow-orange oil. This material was used without purification.

Step 2: The crude diamine from step 1 was combined with CH₂Cl₂ (75 mL), triethylamine (2.67 mL, 19.2 mmol) and benzyl chloroformate (2.71 mL, 19.2 mmol) and stirred at rt for 18 h. The reaction mixture was washed with sat. aq. NaHCO₃ and brine, dried (Na₂SO₄) and concentrated to give an orange oil (5.51 g). Chromatography, flushing first with 50% EtOAc/hexanes and then eluting with 10% MeOH/EtOAc, afforded 1.63 g (32%) of the title compound as a yellow oil: NMR (CDCl₃) δ 7.40-7.14 (m, 9H), 5.20-5.11 (m, 2H), 3.79-3.65 (m, 4H), 2.90 (hept, J=6.8 Hz, 1H), 2.37-2.23 (m, 1H), 2.18-2.05 (m, 1H), 1.70 (br s, 2H), 1.24 (d, J=6.6 Hz, 6H).

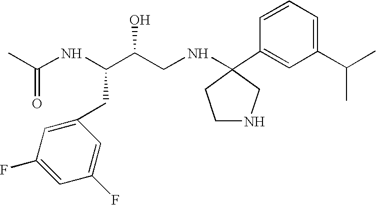

### Preparation 21

### N-{(1S,2R)-1-(3,5-Difluoro-benzyl)-2-hydroxy-3-[3-(3-isopropyl-phenyl)-pyrrolidin-3-ylamino]-propyl}-acetamide

This compound was prepared as a 1:1 mixture of diastereomers using 3-amino-3-(3-isopropyl-phenyl)-pyrrolidine-1-carboxylic acid benzyl ester (Preparation 20) following the general reaction sequence described in Preparations 13-15: NMR (CDCl₃) δ 7.25-7.21 (m, 1H), 7.11-7.00 (m, 3H), 6.67-6.53 (m, 3H), 5.99-5.90 (overlapping doublets, 1H), 4.14-3.99 (m, 1H), 3.33-3.26 (m, 3H), 3.12-2.96 (m, 3H), 2.85-2.78 (m, 3H), 2.74-2.65 (m, 2H), 2.38-2.22 (m, 2H), 2.15-2.05 (m, 2H), 1.83-1.82 (overlapping singlets, 3H), 1.23-1.19 (overlapping doublets, 6H); LCMS MH⁺ observed 446.1, expected 446.3

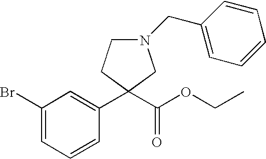

### Preparation 22

### 1-Benzyl-3-(3-bromo-phenyl)-pyrrolidine-3-carboxylic acid ethyl ester

Step 1:

A solution of 2-bromophenyl acetic acid (25.0 g, 116 mmol) in EtOH (200 mL) was saturated with HCl gas. After the mixture had cooled to rt, it was re-saturated with HCl and stirred for 16 h. The reaction was concentrated, dissolved in EtOAc and washed with sat.aq. NaHCO₃ and brine, dried (Na₂SO₄) and concentrated to yield 33.18 g of 3-bromo-phenyl)-acetic add ethyl ester as a yellow oil: NMR (CDCl₃) δ 7.42 (d, J=1.3 Hz, 1H), 7.39-7.36 (m, 1 h), 7.21-7.14 (m, 2H), 4.13 (q, J=7.1 Hz, 2H), 3.55 (s, 2H), 1.23 (t, J=7.3 Hz, 3H).

Step 2:

3-Bromo-phenyl)-acetic acid ethyl ester (5.0 g, 20.6 mmol), sodium ethoxide (1.96 g, 28.8 mmol) and diethyloxalate (5.8 mL, 42.7 mmol) in ether (50 mL) were refluxed for 3 h, diluted with ether, washed with 1N HCl, water and brine, dried (MgSO₄) and concentrated to yield 8.97 g of ˜80% pure 2-(3-bromo-phenyl)-3-oxo-succinic acid diethyl ester as a yellow oil. This was mixed with 37% aq. formaldehyde (4.2 mL, 51.8 mmol) and water (25 mL) and cooled in ice. With vigorous stirring, K₂CO₃ (5.0 g, 36.2 mmol) was added in small portions over ˜2 min. After stirring for 1 h, the reaction was diluted with water (100 mL) and extracted into ether (2×75 mL). The extracts were washed with brine, dried (MgSO₄) and concentrated to give 4.89 g of crude 2-(3-bromo-phenyl)-acrylic acid ethyl ester as a yellow oil. This material and N-(methyloxymethyl)-N-(trimethylsilylmethyl)-benzyl amine (4.9 mL, 19.15 mmol) were dissolved in CH₂C6 (50 mL) and cooled to −10° C. 1N Trifluoroacetic acid in CH₂Cl₂ (0.96 mL, 0.96 mmol) was added and the mixture was allowed to slowly warn to rt and stir for 16 hrs. The reaction was concentrated, redissolved in EtOAc and washed with sat. aq. NaHCO₃ and brine, dried (MgSO₄) and concentrated to yield 7.59 g of a yellow oil. Chromatography, flushing first with 3% EtOAc/hexanes and then eluting with 10% EtOAc/hexanes, afforded 4.75 g of the title compound as a yellow oil: NMR (CDCl₃) δ 7.42 (t, J=1.7 Hz, 1H), 7.34-7.10 (m, 8H), 4.19-4.01 (m, 2H), 3.70 (d, J=13.3 Hz, 1H), 3.60 (d, J=13. Hz, 1H), 3.49 (d, J=8.7 Hz, 1H), 3.02-2.86 (m, 2H), 2.68 (d, J=9.1 Hz, 1H), 2.62-2.50 (sym. mult., 1H), 2.09-2.00 (m, 1H), 1.16 (t, J=7.1 Hz, 3H).

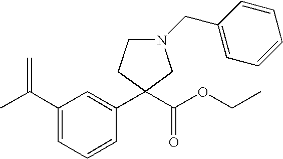

### Preparation 23

### 1-Benzyl-3-(3-isopropenyl-phenyl)-pyrrolidine-3-carboxylic acid ethyl ester

1-Benzyl-3-(3-bromo-phenyl)-pyrrolidine-3-carboxylic acid ethyl ester (Preparation 22, 4.75 g, 12.2 mmol), potassium isopropenyltrifluoroborate (Molander, et al., Org. Lett., 2003, 4(1), 107-9) (2.7 g, 18.2 mmol), triethylamine (2.0 mL, 14.3 mmol), Pd(dppf)Cl₂. CH₂Cl₂ (0-50 g, 0.61 mmol) and n-propanol (200 mL) were heated at 90° C. for 18 h, cooled and concentrated. The residue was partitioned between EtOAc and water, the organics were washed with brine, dried (MgSO₄) and concentrated to afford 4.99 g of a red oil. Chromatography with 5-10% EtOAc/hexanes yielded 2.86 g (67%) of the title compound as a light orange oil: NMR (CDCl₃) δ 742-7.10 (m, 9H), 5.30 (d, J=0.9 Hz, 1H), 5.05-5.04 (m, 1H), 4.25-4.03 (m, 3H), 3.72-3.48 (m, 3H), 3.04-2.94 (m, 1H), 2.92-2.88 (m, 1H), 2.73-2.66 (m, 1H), 2.62-2.52 (m, 1H), 2.10 (s, 3H), 1.15 9t, J=7.1 Hz, 3H).

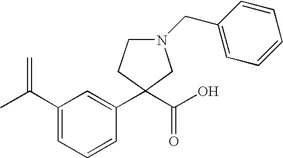

### Preparation 24

### 1-Benzyl-3-(3-isopropenyl-phenyl)-pyrrolidine-3-carboxylic acid

1-Benzyl-3-(3 isopropenyl-phenyl)-pyrrolidine-3-carboxylic acid ethyl ester (Preparation 23, 2.86 g, 8.18 mmol) and lithium hydroxide mono-hydrate (1.7 g, 40.5 mmol) in methanol (30 mL) and water (30 mL) were heated at 70° C. for 17 h, then cooled and concentrated to dryness. This material was cooled in ice, EtOAc (15 mL) and ether (20 mL) were added and 6N HCl (6.75 mL, 40.5 mmol) was added dropwise over 5 min with vigorous stirring. After ˜15 min of additional stirring, a thick orange oil precipitated. The solvent were decanted off and another 2-3 mL water and 20 mL ether were added. This mixture was stirred vigorously for 1 h to form a white solid which was collected, rinsed with ether and dried to yield 2.20 g (85%) of the title compound: NMR (DMSO-d₆) δ 7.48-7.09 (m, 9H), 5.36 (s, 1H), 5.08 (t, J=1.7 Hz, 1H), 3.80-2.50 (very broad multiplets, 8H), 2.05 (s, 3H).

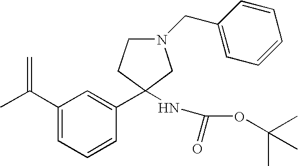

### Preparation 25

### [1-Benzyl-3-(3 isopropenyl-phenyl)-pyrrolidin-3-yl]-carbamic acid tert-butyl ester

1-Benzyl-3-(3-isopropenyl-phenyl)-pyrrolidine-3-carboxylic acid (Preparation 24, 2.20 g, 6.84 mmol) and triethylamine (0.95 mL, 6.82 mmol) in freshly distilled t-butanol were heated to 100° C. to give a hazy yellow solution. After cooling to rt, diphenyl phosphoryl azide (1.5 mL, 6.96 mmol) was added and the mixture was then refluxed for 16 hrs, cooled and concentrated. The residue was partitioned between EtOAc and water, the organics were washed with brine, dried (MgSO₄) and concentrated to afford 3.29 g of sticky orange gum. Chromatography with 10-30% EtOAc/hexanes yielded 1.03 g, (39%) of the title compound as a nearly colorless oil which solidified to a white solid upon evacuation: NMR (CDCl₃) δ 755-7.50 (m, 1H), 7.34-7.12 (m, 8H), 5.31 (t, J=0.9 Hz, 1H), 5.18 (br s, 1H), 5.04 (t, J=1.6 Hz, 1H), 3.72 (br d, J=12.4 Hz, 1H), 3.62-3.56 (m, 1H), 3.00-2.70 (m, 4H), 2.47-2.15 (m, 2H), 2.11 (d, J=0.8 Hz, 3H), 1.36 (br s, 9H).

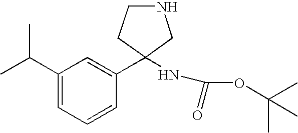

### Preparation 26

### [3-(3-isopropyl-phenyl)pyrrolidin-3-yl]-carbamic acid tert-butyl ester

[1-Benzyl-3-(3-isopropenyl-phenyl)-pyrrolidin-3-yl]-carbamic acid tert-butyl ester (Preparation 25, 1.00 g, 2.55 mmol) was shaken with 10% palladium on carbon (150 mg) under 45 psi of hydrogen for 16 h, filtered (Celite) and concentrated to afford 0.815 g of the title compound as a dirty gray, oily foam: NMR (CDCl₃) δ 7.37-7.08 (m, 4H), 5.03 (br s, 1H), 3.55 (br s, 1H), 3.37-3.13 (m, 3H), 2.87 (hept, J=6.9 Hz, 1H), 2.70-2.00 (br m, 6H), 1.37 (br s, 9H), 1.22 (d, J=7.1 Hz, 6H).

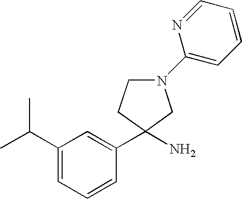

### Preparation 27

### 3-(3-isopropyl-phenyl)-1-pyridin-2-yl-pyrrolidin-3-ylamine

Step 1:

[3-(3-Isopropyl-phenyl)-pyrrolidin-3-yl]-carbamic acid tert-butyl ester (Preparation 26, 0.075 g, 0.246 mmol), 2-bromopyridine (0.028 mL, 0.294 mmol), XANTPHOS (9 mg, 0.015 mmol), Pd₂(dba)₃ (5 mg, 0.0055 mmol) and sodium t-butoxide (0.035 g, 0.312 mmol) in toluene were heated at 100° C. in a sealed tube for 2 h. The reaction was cooled, diluted with EtOAc and washed with water and brine, dried (MgSO₄) and concentrated to afford a yellow oil (100 mg), Chromatography with 10-20% EtOAc/hexanes yielded 50 mg (53%) of [3-(3-isopropyl-phenyl)-1-pyridin-2-yl-pyrrolidin-3-yl]-carbamic add tert-butyl esteras a yellow oil: NMR (CDCl₃) δ 8.15 (dd, J=5.0, 1.2 Hz, 1H), 7.46-7.41 (m, 1H), 7.25-7.18 (m, 3H), 7.10 (dt, J=7.1, 1.7 Hz, 1H), 6.55 (dd, J=6.6, 5.2 Hz, 1H), 6.37 (d, J=8.3 Hz, 1H), 5.12 (br s, 1H), 3.91 (br d, J=10.8 Hz, 1H), 3.82 (br d, J=10.9 Hz, 1H), 3.67-3.47 (m, 2H), 2.89-2.60 (m, 2H), 2.43-2.36 (m, 1H), 1.32 (br s, 9H), 1.19 (d, J=7.1 Hz, 6H).

Step 2:

The compound from step 1 was dissolved in dioxane (1 mL), 4N HCl/dioxane (1 mL) was added and the mbcture was stirred for 17 hrs. A yellow precipitate formed—this was collected and then partitioned between EtOAc and aq. K₂CO₃. The organics were washed with brine, dried (MgSO₄) and concentrated to afford 26 mg (70%) of ˜90% pure title compound as a colorless oil: NMR (CDCl₃) δ 8.17-8.15 (m, 1H), 7.46-7.41 (m, 1H), 7.33-7.23 (m, 3H), 7.15-7.12 (m, 1H), 6.53 (dd, J=6.2, 5.0 Hz, 1H), 6.38 (d, J=8.7 Hz, 1H), 3.84-3.61 (m, 4H), 2.90 (hept, J=7.0 Hz, 1H), 2.45-2.37 (m, 1H), 2.27-2.22 (m, 1H), 1.75 (br s, 2H), 1.23 (d, J=6.6 Hz, 6H).

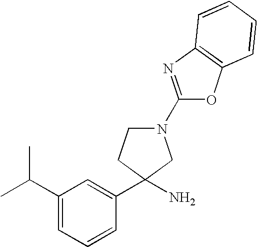

### Preparation 28

### 1-Benzooxazol-2-yl-3-(3-isopropyl-phenyl)-pyrrolidin-3-ylamine

Step 1:

3-(3-Isopropyl-phenyl)-pyrrolidin-3-yl]-carbamic acid tert-butyl ester (Preparation 26, 0.095 g, 0.312 mmol), 2-chlorobenzoxazole (0.040 mL, 0.350 mmol) and K₂HPO₄ (0.085 g, 0.488 mmol) in DMSO (1 mL) were heated to 80° C. in a sealed tube for 2 h, cooled, diluted with water and extracted twice with ether. The extracts were washed with brine, dried (MgSO₄) and concentrated onto silica gel. Chromatography with 5-25% EtOAc/hexanes provide 80 mg (61%) of [1-benzooxazol-2-yl-3-(3-isopropyl-phenyl)-pyrrolidin-3-yl]-carbamic acid tert-butyl ester as a waxy white solid: NMR (CDCl₃) δ 7.36 (d, J=7.9 Hz, 1H), 7.27-7.08 (m, 6H), 7.00 (dt, J=7.7, 1.1 Hz, 1H), 5.09 (br s, 1H), 4.15 (br s, 1H), 4.03 (br s, 2H), 3.89-3.70 (m, 2H), 2.87 (hept, J=6.8 Hz, 1H), 2.44-2.36 (m, 1H), 1.31 (br s, 9H), 1.20 (d, J=6.6 Hz, 6H).

Step 2:

The solid from step 1 was dissolved in dioxane (2 mL) and 4N HCl/dioxane (2 mL) was added and the mixture was stirred for 17 hrs. A white precipitate formed—this was collected and then partitioned between EtOAc and aq. K₂CO₃. The organics were washed with brine, dried (MgSO₄) and concentrated to afford 40 mg (66%) of ˜85% pure title compound as a colorless oil: NMR (CDCl₃) δ 7.35 (d, J=7.9 Hz, 1H), 7.31-7.21 (m, 4H), 7.17-7.11 (m, 2H), 6.98 (dt, J=7.9, 1.3 Hz, 1H), 4.00-3.86 (m, 4H), 2.90 (hept, J=6.9 Hz, 1H), 2.46-2.38 (m, 1H), 2.29-2.23 (m, 1H), 1.75 (br s, 2H), 1.24 (d, J=7.1 Hz, 6H).

### Preparation 29

The following compounds were prepared following the general procedure described in Preparation 28:

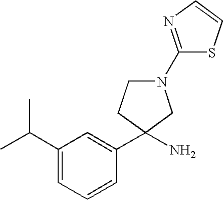

### Preparation 29a

3-(3-Isopropyl-phenyl)-1-thiazol-2-yl-pyrrolidin-3-ylamine: NMR (CDCl₃) δ 7.30-7.21 (m, 3H), 7.19 (d, J=3.7 Hz, 1H), 7.16-7.12 (m, 1H), 6.47 (d, J=3.3 Hz, 1H), 3.84 (d, J=10.4 Hz, 1H), 3.82-3.73 (m, 2H), 3.64 (dt, J=9.1, 2.5 Hz, 1H), 2.89 (hept, J=6.8 Hz, 1H), 2.49-2.41 (m, 1H), 2.29-2.23 (m, 1H), 1.74 (br s, 2H), 1.23 (d, J=7.1 Hz, 6H).

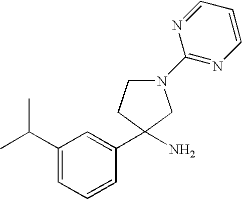

### Preparation 29b

3-(3-Isopropyl-phenyl)-1-pyrimidin-2-yl-pyrrolidin-3-ylamine: NMR (CDCl₃) δ 8.31 (d, J=4.6 Hz, 2H), 7.30-7.23 (m, 3H), 7.14-7.11 (m, 1H), 647 (t, J=4.8 Hz, 1H), 3.95 (dd, J=11.2, 1.7 Hz, 1H), 3.85-3.80 (m, 3H), 2.88 (hept, J=7.5 Hz, 1H), 2.42-2.34 (m, 1H), 2.27-2.22 (m, 1H), 1.71 (br s, 2H), 1.22 (d, J=7.1 Hz, 6H).

What is claimed is:

1. A compound of Formula I: 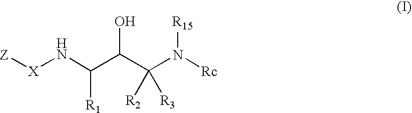
wherein.
Z is hydrogen, (C₃-C₇ cycloalkyl)₀₋₁(C₁-C₆ alkyl)-, (C₃-C₇ cycloalkyl)₀₋₁(C₂-C₆ alkenyl), (C₃-C₇ cycloalkyl)₀₋₁(C₂-C₆ alkynyl), —O—(C₁-C₆)alkyl, —O—(C₂-C₆)alkenyl, —(C₁-C₆) alkyl(C₆-C₁₀)aryl, —(C₂-C₆) alkylene(C₆-C₁₀)aryl or (C₃-C₇ cycloalkyl)-, wherein each of said groups is independently optionally substituted with 1, 2 or 3 R_{Z} groups;
wherein R_{Z} at each occurrence is independently halogen, —OH, —SH, —CN, —CF₃, —OCF₃, (C₁-C₆)alkoxy, (C₃-C₇)cycloalkyl, (C₃-C₇)cycloalkoxy or —NR₁₀₀R₁₀₁;
where R₁₀₀ and R₁₀₁ are independently H, (C₁-C₆)alkyl, phenyl, CO(C₁-C₆)alkyl or SO₂ (C₁-C₆)alkyl;
wherein X is —(C═O— or —(SO₂)—;
wherein R₁ is (C₁-C₁₀)alkyl optionally substituted with 1, 2 or 3 groups independently selected from halogen, —OH, ═O, —SH, —CN, —CF₃, —OCF₃, —(C₃-C₇)cycloalkyl, —(C₁-C₄)alkoxy, —NR₁₀₀R₁₀₁, (C₆-C₁₀)aryl, (5 to 9 member) heteroaryl and (5 to 9 member) heterocyclo, wherein each aryl group is optionally substituted with 1, 2 or 3 R₅₀ groups;
wherein R₅₀ is selected from halogen, OH, SH, CN, —CO—(C₁-C₄)alkyl, —NR₇R₈, —(O)₁₋₂—(C₁-C₄ alkyl), (C₁-C₆)alkyl, (C₂-C₆)alkenyl, (C₂-C₆)alkynyl, (C₁-C₆)alkoxy and (C₃-C₆) cycloalkyl; wherein the alkyl, alkenyl, alkynyl, alkoxy and cycloalkyl groups are optionally substituted with 1 or 2 substituents independently selected from the group consisting of (C₁-C₄)alkyl, halogen, OH, —NR₅R₆, CN, (C₁-C₄)haloalkoxy, NR₇R₈ and (C₁-C₄)alkoxy;
wherein R₅ and R₆ are independently H or (C₁-C₆)alkyl; or
wherein R₅ and R₆ and the nitrogen to which they are attached form a 5 or 6 membered heterocycloalkyl ring; and
wherein R₇ and R₈ are independently selected from the group consisting of H; —(C₁-C₄)alkyl optionally substituted with 1, 2, or 3 groups independently selected from the group consisting of —OH, —NH₂, and halogen; —(C₃-C₆)cycloalkyl; —(C₁-C₄ alkyl)-O—(C₁-C₄ alkyl); —(C₂-C₄)alkenyl; and —(C₂-C₄)alkynyl;
wherein each heteroaryl of R₁ is optionally substituted with 1 or 2 R₅₀ groups; wherein each heterocyclo group of R₁ is optionally substituted with 1 or 2 groups that are independently R₅₀ or ═O;
wherein R₂ and R₃ are independently selected from H; —F; —(C₁-C₆)alkyl optionally substituted with a substituent selected from the group consisting of —F, —OH, —CN, —CF₃, (C₁-C₃)alkoxy and —NR₅R₆; or R₂ and R₃ are independently selected from —(CH₂)₀₋₂—R₁₇; —(CH₂)₀₋₂—R₁₈; —(C₂-C₆)alkenyl or —(C₂-C₆)alkynyl, wherein each group is optionally substituted with a substituent selected from the group consisting of —F, —OH, —CN, —CF₃, (C₁-C₃)alkoxy; —(CH₂)₀₋₂—(C₃-C₇)cycloalkyl, optionally substituted with an independent substituent selected from the group consisting of —F, —OH, —CN, —CF₃, (C₁-C₃)alkoxy and —NR₅R₆; or
wherein R₂, R₃ and the carbon to which they are attached form a —(C₃-C₇)cycloalkyl ring of three through seven carbon atoms, wherein one carbon atom is optionally replaced by a group selected from —O—, —S—, —SO₂— or —NR₇—;
where R₁₇ at each occurrence is an aryl group selected from phenyl, 1-naphthyl, 2-naphthyl, indanyl, indenyl, dihydronaphthyl and tetralinyl, wherein said aryl groups are optionally substituted with one or two groups that are independently —(C₁-C₃)alkyl; —(C₁-C₄)alkoxy; CF₃; or R₁₇ at each occurrence is —C₂-C₆)alkenyl or —(C₂-C₆) alkynyl each of which is optionally substituted with one substituent selected from the group consisting of F, OH, (C₁-C₃)alkoxy; or R₁₇ at each occurrence is selected from
-halogen;
—OH;
—CN;
—(C₃-C₇)cycloalkyl;
—CO—(C₁-C₄ alkyl);
—SO₂—(C₁-C₄ alkyl);
where R₁₈ is a heteroaryl group selected from pyridinyl, pyrimidinyl, quinolinyl, indolyl, pyridazinyl, pyrazinyl, isoquinolyl, quinazolinyl, quinoxalinyl, phthalazinyl, imidazolyl, isoxazolyl, oxazolyl, thiazolyl, furanyl, thienyl, pyrrolyl, oxadiazolyl or thiadiazolyl, wherein each of said heteroaryl groups is optionally substituted with one or two groups that are independently —(C₁-C₆)alkyl optionally substituted with one substituent selected from the group consisting of OH, CN, CF₃, (C₁-C₃)alkoxy and —NR₅R₆;
wherein Rc is 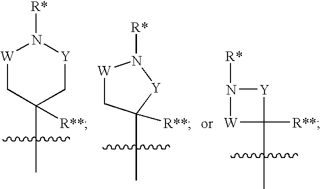
wherein W and Y are each independently —CH₂— or C═O;
wherein R* is 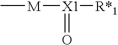
wherein M is —(CH₂)p- or C═O; X1 is C, S═O or is absent; p is 0-3; with the proviso that when M is C═O, X1 is C;
wherein R*₁ is hydrogen, (C₁-C₆)alkyl, (C₁-C₆)alkoxy, hydroxyl (C₁-C₆) alkyl optionally substituted with up to three halogens or OH groups; phenyl; (5 to 9 member)heteroaryl and (5 to 9 member) heterocyclo wherein said heteroaryl is selected from the group consisting of thiazolyl, oxazolyl, 1,2,4-oxadiazolyl, 1,3,4- and 1,2,4-thiadiazolyl, imidazolyl, isoxazolyl, pyridinyl, pyrimidinyl wherein said heteroaryl or heterocyclo is optionally substituted by halogen, (C₁-C₆)alkyl, (C₁-C₆) alkoxy, (C₁-C₆) alkoxyCH₂—, CN, NO₂, CF₃, —NH(C₁-C₆) alkyl, —NH₂, (C₁-C₆)alkyl —CO—NH— or (C₆-C₁₀)aryl(C₁-C₆)alkoxy; said phenyl optionally substituted with (C₁-C₆)alkyl or up to three —(C═O)R*₅ wherein R*₅ is H, (C₁-C₆)alkyl or OH, NR*₂R*₃ or (C═O)(O)₀₋₁R*₄, wherein R*₂, R*₃ and R*₄ are each independently H or (C₁-C₆)alkyl; and
wherein RF is (C₆-C₁₀)aryl, (C₅-C₉)heteroaryl, (C₁-C₁₀)alkyl, (C₂-C₆) alkenyl, (C₂-C₆) alkynyl, —O—(C₁-C₆) alkyl, —O—(C₂-C₆) alkenyl or —(C₂-C₈) alkylene-(C₆-C₁₀)aryl;
wherein each (C₆-C₁₀)aryl of R** is phenyl or naphthyl, each (5 to 9 member) heteroaryl ring is optionally fused to a benzo group and contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₆) alkyl, chloro, bromo-, iodo, fluoro-, (C₁-C₆)hydroxyalkyl-, (C₁-C₆)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, (3-8 membered)heterocyclo, hydroxyl(3-8 membered)heterocyclo and (C₁-C₈)alkoxy-(3-8 membered)heterocyclo, wherein said alkyl, alkoxy and cycloalkyl may be optionally substituted with 1 to 3 halos and wherein each (C₃-C₈)cycloalkyl or heterocyclo moiety may be independently substituted with from one to three (C₁-C₆)alkyl, phenyl or benzyl groups; or
wherein each (C₅-C₉)heteroaryl ring of R** is optionally fused to an imidazo, pyrido, pyrimido, pyrazo, pyridazo, or pyrrolo group and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing fused heteroaryl rings may optionally be substituted with from one to three substituents independently selected from (C₁-C₈) alkyl, chloro-, bromo-, iodo, fluoro-, halo(C₁-C₈)alkyl, hydroxy(C₁-C₈)alkyl-, (C₁-C₈)alkoxy-(C₁-C₈)alkyl-, —(C₁-C₈)alkyl-halo, hydroxy(C₃-C₈)cycloalkyl-, (C₃-C₈)cycloalkoxy-, (C₁-C₈)alkoxy-(C₃-C₈)cycloalkyl-, (5 to 9 member)heterocyclo, hydroxyl (5 to 9 member) heterocyclo and (C₁-C₈)alkoxy-(5 to 9 member)heterocyclo, wherein each (C₃-C₈)cycloalkyl or heterocyclo moiety may be independently substituted with from one to three (C₁-C₆)alkyl or benzyl groups;
with the proviso that when X is —(C═O), Z is methyl, R₁ is difluorobenzyl and R₂ and R₃ are each hydrogen, Rc is 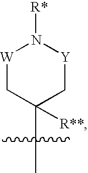
and when
W and Y are each —CH₂ and R** is (C₃-C₄)alkyl substituted phenyl,
R* in combination with M, X1 and R*₁ may not be H, —C₂H₅, (—CH₃)₂, —C₂H₄OH, —C₂H₄CN, —(C═O)NH₂, CH₃—SO₂—, C₂H₅—SO₂—, —H(C═O), —(C═O)CH₃ or —(C═O)CF₃;
with the additional proviso that when X is —(C═O), Z is methyl, R₁ is difluorobenzyl and R₂ and R₃ are each hydrogen, Rc is 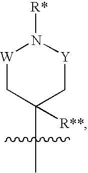
and when
W and Y are each —CH₂— and R** is (C₃-C₄)alkyl substituted phenyl, R* in combination with M, X1 and R*₁ may not be (C₁-C₃) alkyl, hydroxyl (C₁-C₃) alkyl, —CN(C₁-C₃)alkyl, —(C═O)NR₇R₈, (C₁-C₃)alkyl-SO₂—, —(C₁-C₃)alkyl-CHO, —(C═O)(C₁-C₃) alkyl or —(C═O)(C₁-C₃) alkyl wherein said alkyl is substituted with 3-5 fluoro atoms.

2. The compound of claim 1 having the Formula Ia 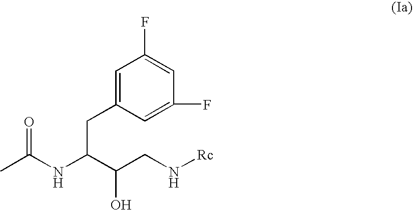
wherein Rc has the same meaning as defined above.

3. The compound of claim 2 wherein Rc is 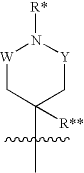
wherein each of W, Y, R* and R** has the same meaning as defined above.

4. The compound of claim 3 wherein W and Y are each —CH₂—; R* is —COOCH₃ or —COO—CH₂-phenyl; and R** is (C₁-C₄)alkyl substituted phenyl.

5. The compound of claim 3 wherein W is —CH₂—, and Y is C═O; R* is H, (C₁-C₄)alkyl, —CH₂COOH, —CH₂COOCH₃, —CH₂COOCH(CH₃)₂, CH₃—CH₂-phenyl or —CH₂—CH₂OH; and R** is C₁-C₄ alkyl substituted phenyl.

6. The compound of claim 5 wherein the carbon atom at the piperidone ring 4 position is a chiral carbon atom in the S configuration.

7. The compound of claim 3 wherein W and Y are each C═O.

8. The compound of claim 2 wherein Rc is 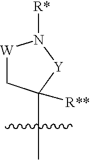
wherein W and Y are each —CH₂—, R** is (C₃-C₄)alkyl substituted phenyl and R* is 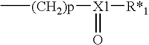
wherein p is 0, X1 is C and R*₁ is —CH₃, —OCH₃ or —CH₂—CO—OCH₃.

9. The compound of claim 8 wherein X1 is absent and R*₁ is heteroaryl.

10. The compound of claim 9 wherein R*₁ is 2-thiazolyl or 2-pyrimidinyl

11. The compound of claim 2 wherein Rc is 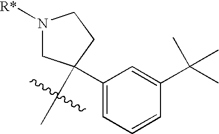
wherein R* is 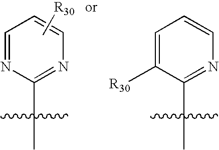
wherein R₃₀ is (C₁-C₆)alkyl, (C₁-C₆) alkoxy, (C₁-C₆)alkylene-O—(C₁-C₆)alkyl, CN, NH₂, NH(C₁-C₆)alkyl, CF₃, NO₂ or halogen.

12. A pharmaceutical composition comprising the compound of claim 1 and a pharmaceutically acceptable carrier.

13. A compound of claim 1 selected from the group consisting of
4-[(2R,3S)-3-Acetylamino-4-(3,5-difluoro-phenyl)-2-hydroxy-butylamino]-4-(3-isopropyl-phenyl)-piperidine-1-carboxylic acid methyl ester;
N-{(1S,2R)-1-(3,5-Difluoro-benzyl)-2-hydroxy-3-[1-(2-hydroxy-acetyl)-4-(3-isopropyl-phenyl)-piperidin-4-ylamino]-propyl}acetamide;
[(2R,3S)-4-[3-Acetylamino-4-(3,5-difluoro-phenyl)-2-hydroxy-butylamino]-4-(3-isopropyl-phenyl)-piperidin-1-yl]-oxo-acetic acid;
[(2R,3S)-4-[Acetylamino-4-(3,5-difluoro-phenyl)-2-hydroxy-butylamino]-4-(3-isopropyl-phenyl)-piperidin-1-yl]-oxo-acetic acid methyl ester;
N-{(1S,2R)-1-(3,5-Difluoro-benzyl)-2-hydroxy-3-[4-(3-isopropyl-phenyl)-1-(2,2,2-trifluoro-ethanesulfonyl)-piperidin-4-ylamino]-propyl}-acetamide;
4-[(2R,3S)-3-Acetylamino-4-(3,5-difluoro-phenyl)-2-hydroxy-butylamino]-4-(3-isopropyl-phenyl)-piperidine-1-carboxylic acid methylamide;
4-[(2R,3S)-3-Acetylamino-4-(3,5-difluoro-phenyl)-2-hydroxy-butylamino]-4-(3-isopropyl-phenyl)-piperidine-1-carboxylic acid ethyl ester;
4-[(2R,3S)-3-Acetylamino-4-(3,5-difluoro-phenyl)-2-hydroxy-butylamino]-4-(3-tert-butyl-phenyl)-piperidine-1-carboxylic acid benzyl ester;
4-[(2R,3S)-3-Acetylamino-4-(3,5-difluoro-phenyl)-2-hydroxy-butylamino]-4-(3-isopropyl-phenyl)-piperidine-1-carboxylic acid benzyl ester;
4-[(2R,3S)-3-Acetylamino-4-(3,5-difluoro-phenyl)-2-hydroxy-butylamino]-4-(3-tert-butyl-phenyl)-piperidine-1-carboxylic acid methyl ester;
N-[(1S,2R)-3-[4(3-tert-Butyl-phenyl)-1-butyryl-piperidin-4-ylamino]-1-(3,5-difluoro-benzyl)-2-hydroxy-propyl]-acetamide;
N-[(1S,2R)-3-[4(3-tert-Butyl-phenyl)-1-(3-methyl-butyryl)-piperidin-ylamino]-1-(3,5-difluoro-benzyl)-2-hydroxy-propyl]-acetamide;
4-[(2R,3S)-3-Acetylamino-4-(3,5-difluoro-phenyl)-2-hydroxy-butylamino]-4-(3-tert-butyl-phenyl)-piperidine-1-carboxylic acid dimethylamide;
[(2R,3S)-4-[3-Acetylamino-4-(3,5-difluoro-phenyl)-2-hydroxy-butylamino]-4-(3-isopropyl-phenyl)-2-oxo-piperidin-1-yl]-acetic acid methyl ester;
[(2R,3S,4R)-4-[3-Acetylamino-4-(3,5-difluoro-phenyl)-2-hydroxy-butylamino]-4-(3-isopropyl-phenyl)-2-oxo-piperidin-1-yl]-acetic acid methyl ester;
[(2R,3S)-4-[3-Acetylamino-4-(3,5-difluoro-phenyl)-2-hydroxy-butylamino]-4-(3-isopropyl-phenyl)-2-oxo-piperidin-1-yl]-acetic acid;
[(2R,3S,4R)-4-[3-Acetylamino-4-(3,5-difluoro-phenyl)-2-hydroxy-butylamino]-4-(3-isopropyl-phenyl)-2-oxo-piperidin-1-yl]-acetic add;
[(2R,3S,4S)-4-[3-Acetylamino-4-(3,5-difluoro-phenyl)-2-hydroxy-butylamino]-4-(3-isopropyl-phenyl)-2-oxo-piperidin-1-yl]-acetic acid methyl ester;
[(2R,3S,4S)-4-[3-Acetylamino-4-(3,5-difluoro-phenyl)-2-hydroxy-butylamino]-4-(3-isopropyl-phenyl)-2-oxo-piperidin-1-yl]-acetic acid;
N-{(1S,2R)-1-(3,5-Difluoro-benzyl)-3-[1-ethyl-4-(3-isopropyl-phenyl)-2-oxo-piperidin-4-ylamino]-2-hydroxy-propyl}-acetamide;
N-{(1S,2R,4R)-1-(3,5-Difluoro-benzyl)-3-[1-ethyl-4-(3-isopropyl-phenyl)-2-oxo-piperidin-4-ylamino]-2-hydroxy-propyl}-acetamide;
N-{(1S,2R,4S)-1-(3,5-Difluoro-benzyl)-3-[1-ethyl-4-(3-isopropyl-phenyl)-2-oxo-piperidin-4-ylamino]-2-hydroxy-propyl}acetamide;
N-[(1S,2R)-3-[1-Benzyl-4-(3-isopropyl-phenyl)-2-oxo-piperidin-4-ylamino]-1-(3,5-difluoro-benzyl)-2-hydroxy-propyl]-acetamide;
N-[(1S,2R,4S)-3-[1-Benzyl-4-(3-isopropyl-phenyl)-2-oxo-piperidin-4-ylamino]-1-(3,5-difluoro-benzyl)-2-hydroxy-propyl]-acetamide;
N-[(1S,2R,4R)-3-[1-Benzyl-4-(3-isopropyl-phenyl)-2-oxo-piperidin-ylamino]-1-(3,5-difluoro-benzyl)-2-hydroxy-propyl]-acetamide;
N-{(1S,2R)-1-(3,5-Difluoro-benzyl)-2-hydroxy-341-(2-hydroxy-ethyl)-4-(3-isopropyl-phenyl)-2-oxo-piperidin-4-ylamino}-propyl)acetamide;
N-{(1S,2R,4R)-1-(3,5-Difluoro-benzyl)-2-hydroxy-3-[1-(2-hydroxy-ethyl)-4-(3-isopropyl-phenyl)-2-oxo-piperidin-4-ylamino]-propyl}acetamide;
N-{(1S,2R,4S)-1-(3,5-Difluoro-benzyl)-2-hydroxy-3-[1-(2-hydroxy-ethyl)-4-(3-isopropyl-phenyl)-2-oxo-piperidin-4-ylamino]-propyl}acetamide;
N-{(1S,2R)-1-(3,5-Difluoro-benzyl)-2-hydroxy-3-[4-(3-isopropyl-phenyl)-2-oxo-piperidin-4-ylamino]-propyl}acetamide;
N-{(1S,2R,4S)-1-(3,5-Difluoro-benzyl)-2-hydroxy-3-[4-(3-isopropyl-phenyl)-2-oxo-piperidin-4-ylamino]-propyl}acetamide;
N-{(1S,2R,4R)-1-(3,5-Difluoro-benzyl)-2-hydroxy-3-[4-(3-isopropyl-phenyl)-2-oxo-piperidin-4-ylamino]-propyl}acetamide;
N-(1S,2R)-[3-[1-(2-tert-Butoxy-ethyl)-4-(3-isopropyl-phenyl)-2-oxo-piperidin-4-ylamino]-1-(3,5-difluoro-benzyl)-2-hydroxy-propyl]-acetamide;
N-(1S,2R,4S)-[3-[1-(2-tert-Butoxy-ethyl)-(3-isopropyl-phenyl)-2-oxo-piperidin-4-ylamino]-1-(3,5-difluoro-benzyl)-2-hydroxy-propyl]-acetamide;
N-(1S,2R,4R)-[3-[1-(2-tert-Butoxy-ethyl)-(3-isopropyl-phenyl)-2-oxo-piperidin-4-ylamino]-1-(3,5-difluoro-benzyl)-2-hydroxy-propyl]-acetamide;
(2R,3S)-[4-[3-Acetylamino-(3,5-difluoro-phenyl)-2-hydroxy-butylamino]-4-(3-isopropyl-phenyl)-2-oxo-piperidin-1-yl]-acetic acid isopropyl ester;
(2R,3S,4R)-[4-[3-Acetylamino-(3,5-difluoro-phenyl)-2-hydroxy-butylamino]-4-(3-isopropyl-phenyl)-2-oxo-piperidin-1-yl]-acetic acid isopropyl ester;
(2R,3S,4S)-[4-[3-Acetylamino-4-(3,5-difluoro-phenyl)-2-hydroxy-butylamino]4-(3-isopropyl-phenyl)-2-oxo-piperidin-1-yl]-acetic acid isopropyl ester;
N-[(1S,2R)-3-[1-Acetyl-3-(3-isopropyl-phenyl)-pyrrolidin-3-ylamino]-1-(3,5-difluoro-benzyl)-2-hydroxy-propyl]-acetamide;
3-[(2S,3R)-3-Acetylamino-4-(3,5-difluoro-phenyl)-2-hydroxy-butylamino]-3-(3-isopropyl-phenyl)-pyrrolidine-1-carboxylic acid methyl ester;
3-[(2S,3R)-3-Acetylamino-(3,5-difluoro-phenyl)-2-hydroxy-butylamino]-3-(3-isopropyl-phenyl)-pyrrolidine-1-carboxylic acid methyl ester;
N-{(1S,2R)-1-(3,5-Difluoro-benzyl)-2-hydroxy-3-[3-(3-isopropyl-phenyl)-1-methanesulfonyl-pyrrolidin-3-ylamino]-propyl}-acetamide;
N-{(1S,2R)-1-(3,5-Difluoro-benzyl)-2-hydroxy-3-[3-(3-isopropyl-phenyl)-1-methanesulfonyl-pyrrolidin-3-ylamino]-propyl}acetamide;
3-[(2S,3R)-3-[3-Acetylamino-4-(3,5-difluoro-phenyl)-2-hydroxy-butylamino]-3-(3-isopropyl-phenyl)-pyrrolidin-1-yl]-3-oxo-propionic acid methyl ester;
3-[(2S,3R)-3-[3-Acetylamino-4-(3,5-difluoro-phenyl)-2-hydroxy-butylamino]-3-(3-isopropyl-phenyl)-pyrrolidin-1-yl]-3-oxo-propionic acid methyl ester;
N-{(1S,2R)-1-(3,5-Difluoro-benzyl)-2-hydroxy-3-[3-(3-isopropyl-phenyl)-pyridin-2-yl-pyrrolidin-3-ylamino]-propyl}acetamide;
N-[(1S,2R)-3-[1-Benzooxazol-2-yl-3-(3 isopropyl-phenyl)-pyrrolidin-3-ylamino]-1-(3,5-difluoro-benzyl)-2-hydroxy-propyl]-acetamide;
N-{(1S,2R)-1-(3,5-Difluoro-benzyl)-2-hydroxy-3-[3-(3-isopropyl-phenyl)-1-thiazol-2-yl-pyrrolidin-3-ylamino]-propyl}acetamide;
N-{(1S,2R)-1-(3,5-Difluoro-benzyl)-2-hydroxy-3-[3-(3-isopropyl-phenyl)-1-pyrimidin-2-yl-pyrrolidin-3-ylamino]-propyl}acetamide;
N-[(1S,2R)-3-[1-(5-Bromo-pyrimidin-2-yl)-3-(3-tert-butyl-phenyl)-pyrrolidin-3-ylamino]-1-(3,5-difluoro-benzyl)-2-hydroxy-propyl]-acetamide;
N-[(1S,2R)-3-[3-(3-tert-Butyl-phenyl)-1-(4-methoxy-pyrimidin-2-yl)-pyrrolidin-3-ylamino]-1-(3,5-difluoro-benzyl)-2-hydroxy-propyl]-acetamide;
3-[(2R,3S)-3-Acetylamino-4-(3,5-difluoro-phenyl)-2-hydroxy-butylamino]-3-(3-isopropyl-phenyl)-azetidine-1-carboxylic acid benzyl ester;
3-[3-(2R,3S)-3-Acetylamino-4-(3,5-difluoro-phenyl)-2-hydroxy-butylamino]-3-(3-isopropyl-phenyl)-azetidin-1-yl]-3-oxo-propionic acid methyl ester;
3-[(2R,3S)-3-Acetylamino-4-(3,5-difluoro-phenyl)-2-hydroxy-butylamino]-3-(3-isopropyl-phenyl)-azetidine-1-carboxylic acid methyl ester;
N-[(1S,2R)-3-[1-Acetyl-3-(3-isopropyl-phenyl)-azetidin-3-ylamino]-1-(3,5-difluoro-benzyl)-2-hydroxy-propyl]-acetamide;
N-[(1S,2R)-3-[4(3-tert-Butyl-phenyl)-1-pyrimidin-2-yl-piperidin-4-ylamino]-1-(3,5-difluoro-benzyl)-2-hydroxy-propyl]-acetamide; and
N-[(1S,2R)-3-[4(3-tert-Butyl-phenyl)-1-thiazol-2-yl-piperidin-4-ylamino]-1-(3,5-difluoro-benzyl)-2-hydroxy-propyl]-acetamide.

14. A process for preparing a compound of the formula 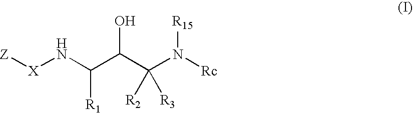
comprising reacting, under conditions effective to form said compound I, a compound of formula 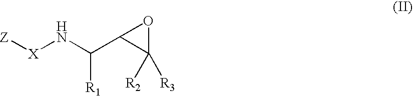
with a compound of formula 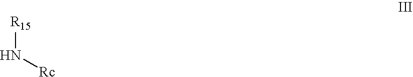
wherein Z, X, R₁, R₂, R₃, Rc and R₁₅ are as defined hereinabove.

15. A pharmaceutical composition comprising the compound of claim 1 and a pharmaceutically acceptable carrier.

16. A method of treatment of a disorder or condition selected from the group consisting of Alzheimer's disease, mild cognitive impairment, Down's syndrome, Hereditary Cerebral Hemorrhage with Amyloidosis of the Dutch-Type, cerebral amyloid angiopathy, other degenerative dementias, dementias of mixed vascular and degenerative origin, dementia associated with Parkinson's disease, dementia associated with progressive supranuclear palsy, dementia associated with cortical basal degeneration, diffuse Lewy body type of Alzheimers disease, the method comprising administering to a mammal in need of such treatment the compound of claim 1.
